(19) United States
(12) Patent Application Publication
(10) Pub. No.: US 2010/0203546 A1
(43) Pub. Date: Aug. 12, 2010

(54) Allele Determining Device, Allele Determining Method And Computer Program

(75) Inventors: Mami Yamashita, Tokyo (JP); Mikio Kawahara, Tokyo (JP)

Correspondence Address: SQUIRE, SANDERS & DEMPSEY L.L.P., 1 MARITIME PLAZA, SUITE 300, SAN FRANCISCO, CA, 94111, US

(73) Assignee: Toppan Printing Co., Ltd., Tokyo (JP)

(21) Appl. No.: 12/764,679

(22) Filed: Apr. 21, 2010

Related U.S. Application Data
(63) Continuation of application No. PCT/JP2008/069274, filed on Oct. 23, 2008.

(30) Foreign Application Priority Data
Oct. 26, 2007 (JP) 2007-279105

(51) Int. Cl. C12Q 1/68; C12M 1/34

(52) U.S. Cl. 435/6; 435/287.2

(57) ABSTRACT

According to an allele determining device 1 that determines a single nucleotide polymorphism of a gene, an approximating unit 4 approximates an optical measurement result obtained by observing a reagent that reacts with a specific base sequence of a gene, to a logistic curve that is a curve using light intensity and time as parameters. A determining unit 5 determines a single nucleotide polymorphism using a plateau value or an inflection point that is a characteristic point of the approximated logistic curve, the measured light intensity, and the like.

### CROSS REFERENCE TO RELATED APPLICATION

This application is a continuation of PCT International Application No. PCT/JP2008/069274, the entire disclosure of which is incorporated herein by reference.

### BACKGROUND OF THE INVENTION

1. Field of the Invention

The present invention relates to an allele determining device, an allele determining method and a computer program of an SNP (single nucleotide polymorphism) utilizing a result of measurement that was optically obtained from a probe for identifying a specific base sequence.

2. Description of the Related Art

Genomes have various kinds of variations, and a genome having 1% or higher variations is observed in a group of certain organism species is called polymorphism. Among the polymorphism, an SNP (single nucleotide polymorphism) is variety in which a single base varies in genome base sequence of a group of certain organism species (called mutation if 1% or less). A human genome includes 3,000,000,000 pair of bases, and it is estimated that one SNP exists in 1,000 by in average. The SNP changes configuration and function of protein, and differentiates individual phenotypes.

In recent years, many SNPs of gene that are expected to be applied in clinical test are found, some relate to medicine metabolism, and some largely influence intensity of effect of medicine. If there is SNP that lowers enzyme activity in gene thereof, blood concentration of the medicine is maintained high for a long time and as a result, effect appears strongly, or harmful intermediate metabolism produce is accumulated in some cases. If there is SNP in which medicine does not work well, it is necessary to increase a dosage amount. Hence, for “custom-made medical care” that is suitable for individual constitutional predispositions by genetic information, it is conceived that SNP of gene is inspected before dosing, and the obtained type of gene is utilized as information for determining a suitable dosage amount of medicine and the like. With this, it is possible to avoid a side effect, to expect that an efficient treatment effect can be obtained, dealing of useless side effect or unsuitable dosing can be reduced, and reduction in cost of medical care can be expected.

For detecting SNP, Restriction Fragment Length Polymorphisms (RFLPs) are conventionally used, but in recent years, various methods such as an Invader (registered trademark) method, a TaqMan PCR method, a single nucleotide primer elongation reaction, an SNaPshot (registered trademark) method, a Pyrosequencing™ method, a Melting Point method, and an SSCP (Single-stranded conformational polymoriphism analysis) which are more simple and general methods are developed.

Referring to FIG. 26, the Invader (registered trademark) method that is one of techniques for determining a genetic pattern of SNP based on a result of measurement that is optically obtained from a probe that identifies a specific base sequence will be described.

In the Invader (registered trademark) method, SNP is detected using the following two-stage reaction. When there is a target base in a targeting portion of a target DNA (Target nucleic acid) that is a DNA from which SNP is to be detected, enzyme called Cleavase (registered trademark) specifically recognizes a ternary complex structure formed by the target DNA, Invader (registered trademark) oligo, or allele oligo that is a signal probe in the first reaction, and a flap portion of allele oligo that did not form a base pair is cut. The allele oligo is an oligo nucleotide constituted by the flap portion and the portion that recognizes the target base sequence. The Invader (registered trademark) oligo is oligo nucleotide that recognizes target base sequence in the target DNA and only one base enters the allele oligo. The Cleavase (registered trademark) is enzyme that recognizes and cuts a structure (invading structure) in which two kinds of oligo are superposed, and is a kind of DNA recovery enzyme. (The Invader method uses a fluorescence reaction including a reaction process using a substrate specificity of an enzyme.)

In the next second reaction, the flap that liberated in the first reaction and FRET™ cassette that is a FRET probe hybridize to form the complex structure. The Cleavase (registered trademark) that is the same enzyme as the first reaction cuts the complex structure, and fluorescent material that is released from fluorescence quenching emits fluorescence. The FRET cassette is a probe including a portion that recognizes a flap fragment caused by the Cleavase (registered trademark), fluorescent material (F), and light quenching material (Q), and is designed such that a flap fragment can enter a sequence between the fluorescent material (F) and the light quenching material (Q). When a distance between the fluorescent material (F) and the light quenching material (Q) is close, the fluorescent material (F) does not emit fluorescence due to light-quenching effect of the light quenching material (Q), but if the fluorescent material (F) liberates due to the Cleavase (registered trademark) and is separated from the light quenching material (Q), the fluorescent material (F) emits fluorescence.

According to the Invader (registered trademark) method, it is possible to simultaneously detect wild type and mutant by two sets of allele oligo with one well (reaction system), an FRET cassette, and Invader (registered trademark) biplex format in which two kinds of fluorescent pigments are put. The wild type is a gene type that is most frequently generated in a natural group in one organism species. On the other hand, when some changes are brought into gene DNA, gene whose heredity is changed is called mutant.

A normal organism includes two alleles from parent. When the same kinds of genes are taken over from the parent, it is called homojunction, and different kinds of genes are taken over, it is called heterojunction. There are three types of SNPs detected by the Invader (registered trademark) method, i. e., wild type homojunction, homojunction of mutant, and heterojunction of wild type and mutant.

According to the Invader (registered trademark) biplex format, wild type gene and mutant gene are detected by two kinds of fluorescent pigments, thereby determining the three kinds of SNP types. For example, if a probe is designed such that flap fragment which allele oligo generates is coupled to an FRET cassette to which fluorescent pigment called FAM is attached, wild type is detected, flap fragment which allele oligo generates is coupled to the FRET cassette to which fluorescent pigment called RED is attached, and mutant is detected, only FAM fluorescence is detected by the wild type homojunction, only RED fluorescence is detected by homojunction of mutant, and both FAM and RED fluorescence is detected by wild type and mutant heterojunction.

Measuring procedure by the Invader (registered trademark) method will be described below.
(1) DNA is extracted by sample such as blood.
(2) DNA sample is amplified by PCR reaction.
(3) Invader (registered trademark) reagent, i. e., various oligo and enzyme Cleavase (registered trademark) are mixed to denatured PCR product.
(4) The mixture is incubated at a constant temperature, and Invader (registered trademark) reaction is carried out.
(5) Two kinds of fluorescence values are measured by a fluorescence measurement device with time. Since wavelength regions that the two kinds of fluorescent pigments emit are different from each other, two kinds of filters for detecting the respective wavelength regions are attached to the fluorescence measurement device, and the fluorescence values are measured.

Non-patent document 1 discloses a method in which a measurement result of real-time RT-PCR is fitted using a sigmoid curve and analyzed.

Non-patent document 1: Hao Qiu et al., “Gene expression of HIF-1 α and XRCC4 measured in human samples by real-time RT-PCR using the sigmoidal curve-fitting method”, Bio Techniques, 2007, Vol. 42, pp. 355-362.

### SUMMARY OF THE INVENTION

FIG. 27 shows examples of patterns obtained as analysis data using the Invader (registered trademark) method as described above. In the Invader (registered trademark) method, probes that are two kinds of fluorescent pigments, i.e., FAM and RED, specifically reacts with a portion where it is desired to check whether there is SNP. When base of that portion is A (adenine), FAM emits fluorescence, and when the base is G (guanine), RED emits fluorescence. When allele is AA, only FAM fluorescence is detected (FAM Homo), and when the allele is GG, only RED is detected (RED Homo), and when the allele is AG, both FAM and RED are detected (Hetero). As shown in FIGS. 27A to B, four patterns are observed as results of the analysis. In these drawings, horizontal axes show time, and vertical axes show fluorescence intensities of FAM and RED. Here, a case where scales of fluorescence intensities of FAM and RED are set at the same level is illustrated, and raw data of FAM and RED that is actually obtained by a device differs by about 5 to 15 times depending upon setting of the device. FIG. 27A shows a case of Hetero, where both FAM and RED are shown with positive reaction curves. FIG. 27B shows a case of FAM Homo, FAM is shown with a positive reaction curve and RED is shown with a negative reaction curve. FIG. 27C shows a case of RED Homo, RED is shown with a positive reaction curve and FAM is shown with negative reaction curve. In the case of NG as shown in FIG. 27D, both FAM and RED are negative reaction curves.

In a conventional technique, gene type of SNP is determined based on a fluorescence intensity ratio between FAM and RED in end point T using these patterns as shown in FIG. 28. FIG. 28A shows fluorescence intensities of FAM and RED at time t, and fluorescence intensity of FAM when time t is equal to the end point T is defined as F^{R}(T), and fluorescence intensity of RED at that time is defined as R^{R}(T). In FIG. 28B, an x-axis shows FAM fluorescence intensity F^{R}(T) in the end point T, a y-axis shows RED fluorescence intensity R^{R}(T), and (F^{R}(T), R^{R}(T)) is plotted. Clusters (aggregation) of RED Homo, Hetero and FAM Homo are formed in boundaries of two straight lines y=ax and y=(1/a)x (in the drawing, a=2). FIG. 28C shows an example where an actual observation result is plotted, and it can be found that it follows cluster division in FIG. 28B.

Specific SNP determining method of the Invader (registered trademark) method is described below. Blood and extracted DNA are used as samples. Examples of kinds of data obtained by measurement are sample data (raw data), corrected data, negative control (NC) data, and positive control (PC) data.

The sample data (raw data) is fluorescence intensity of each of FAM and RED after t-minutes (measured by the device), fluorescence intensity of FAM at time t is defined as F^{A}(t), and fluorescence intensity of RED is defined as R^{A}(t).

The corrected data is sample data corrected by a certain algorithm, data obtained by correcting fluorescence intensity F^{A}(t) of FAM is defined as F^{R}(t), and data obtained by correcting fluorescence intensity R^{A}(t) of RED is defined as R^{R}(t).

The negative control (NC) data is negative control data measured without sample, and data having all reagents except sample is measured. A value of the negative control is varied if a reagent configuration is varied. Fluorescence intensity of FAM of negative control at time t is defined as F^{N}(t), and fluorescence intensity of RED is defined as R^{N}(t).

The SNP determining procedure by the Invader (registered trademark) method using the above data is as follows.

(1) In time t=end point T (e. g., two minutes), FAM fluorescence intensity F^{A}(T) and RED fluorescence intensity R^{A}(T) of sample data, and FAM fluorescence intensity F^{N}(T) and RED fluorescence intensity R^{N}(T) of negative control data are obtained.

(2) Corrected data F^{R}IT), R^{R}(T) are obtained by the following calculation. Here, (F^{A}(T)/F^{N}(T)), (R^{A}(T)/R^{N}(T)) of numerators mean that a negative control value is subtracted, and a denominator means that it makes intensity ratios (scales) of F^{A}(T) and F^{N}(T) match with each other. This is based on the premise that an intensity ratio of a sample value and an intensity ratio of a negative control match with each other.

F^{R}(T)=(F^{A}(T)/F^{N}(T))−1=(F^{A}(T)−F^{N}(T))/F^{N}(T), R^{R}(T)=(R^{A}(T)/R^{N}(T))−1=(R^{A}(T)−R^{N}(T))/R^{N}(T)

(3) A ratio Ratio between corrected data F^{R}(T) and R^{R}(T) are calculated as follows, and allele is determined by a calculation result. If the Ratio<(1/a), it is determined that it is FAM Homo, and if (1/a)<Ratio<a, it is determined that it is Hetero, and if Ratio >a, it is determined that it is RED Homo.

Ratio=R^{R}(T)/F^{R}(T)

However, in the actual measurement, when a target DNA only includes allele in which fluorescence should not normally be detected, a fluorescence value is gradually increased in some cases. This phenomenon is called “rise in background”. It is conceived that the background rises because a specific portion of allele oligo that should not be cut under normal condition is erroneously cut by enzyme Cleavase (registered trademark). Therefore, if the amount of target DNA becomes excessive, the probability that the allele oligo is erroneously cut is increased, and it is said that the background is prone to rise. The rise in the background is a factor that SNP is erroneously determined. A graph in which data where such background rises is plotted is shown in FIG. 29. In the drawings, thick lines show actually measured values, and the values are largely deviated from ideal curves. In FIG. 29A, RED background rises although Homo is FAM Homo, and in FIG. 29B, FAM background rises although Homo is RED Homo.

When the end point method is used, corrected data values F^{R}(T) and R^{R}(T) largely depend on negative control data values F^{N}(T) and R^{N}(T). In the end point method, the sample data is divided by negative control data, and this means that an intensity ratios (scale) of FAM and RED matches with each other. This is on the precondition that an intensity ratio of the sample value “F^{R}(T) : R^{R}(T)” and an intensity ratio of the negative control value “F^{N}(T) : R^{N}(T)” match with each other. However, the negative control data is originally small in value, and an error is prone to be generated. FIG. 30 shows variation with time of NC Ratio=F^{N}(T)/R^{N}(T). As shown in the drawing, actually intensity ratios of both negative control data NC1 and negative control data NC2 are varied (since reagent configurations of the negative control data NC1 and negative control data NC2 are different from each other, the average values thereof are different but there is no problem). Therefore, the actually measured data includes backlash (noise). Therefore, when calculation is directly carried out using the measured data (especially differentiation or division), local noise is largely influenced. As a method for suppressing a local noise, there is a method using a smoothening filter (median filter or the like), but there is a limitation in such method also.

According to the conventional SNP determining method, the above-described rise in the background or noise may cause an erroneous determination. However, the determination of SNP is assumed to be utilized also in the medical field in the feature as described above, it is required to enhance the determining precision.

The present invention has been accomplished in view of the above circumstances, and it is an object of the invention to provide an allele determining device, an allele determining method and a computer program capable of precisely determining SNP (single nucleotide polymorphism).

To achieve the above object, an invention of first aspect of the present invention provides an allele determining device that determines a single nucleotide polymorphism of a gene, including approximating means that approximates an optical measurement result obtained by observing a reagent that reacts with a specific base sequence of a gene, to a predetermined curve using light intensity and time as parameters, and determining means that determines a single nucleotide polymorphism using a characteristic point of the curve that was approximated by the approximating means.

According to a second aspect of an invention, in the allele determining device according to the first aspect, the characteristic point is an inflection point in the curve that was approximated by the approximating means.

According to an third aspect of an invention, in the allele determining device according to the second aspect, the determining means further determines the single nucleotide polymorphism using an index of a maximum value of the light intensity in the curve that was approximated by the approximating means.

According to a fourth aspect of an invention, in the allele determining device according to the second aspect, the determining means determines the single nucleotide polymorphism using a maximum intensity of observed light indicated by the optical measurement result.

According to a fifth aspect of an invention, in the allele determining device according to the first aspect, the approximating means approximates optical measurement results of two kinds of reagents that react with different specific base sequences to the predetermined curve, and the determining means determines whether reactions of the reagents are positive or negative using the characteristic point of the curve that was approximated by the approximating means for each of the reagents, and determines a single nucleotide polymorphism from the determination result.

According to a sixth aspect of an invention, in the allele determining device according to the first aspect, the determining means calculates end point time from the characteristic point, and determines the single nucleotide polymorphism using the optical measurement result observed at the calculated end point time.

According to a seventh aspect of an invention, in the allele determining device according to the sixth aspect, the determining means determines the single nucleotide polymorphism further using a logarithm of a ratio of the optical measurement result at the endpoint time of each of the two kinds of reagents that react with different specific base sequences.

A eighth aspect of an invention provides an allele determining device that determines a single nucleotide polymorphism of a gene, including approximating means that approximates an optical measurement result obtained by observing a reagent that reacts with a specific base sequence of a gene, to a predetermined curve using light intensity and time as parameters, and determining means that determines a single nucleotide polymorphism using a characteristic point obtained from a logarithm of a curve that was approximated by the approximating means.

According to a ninth aspect of an invention, in the allele determining device according to the eighth aspect, the approximating means approximates optical measurement results of two kinds of reagents that react with different specific base sequences to the predetermined curve, and the determining means determines the single nucleotide polymorphism using a characteristic point obtained from a logarithm of a ratio of the curve that was approximated to the reagents by the approximating means.

According to a tenth aspect of an invention, in the allele determining device according to the ninth aspect, the characteristic point is a peak value in the logarithm of the ratio.

According to an eleventh aspect of an invention, in the allele determining device according to the first aspect, the curve is a logistic curve.

According to a twelfth aspect of an invention, in the allele determining device according to the first aspect, the optical measurement result is a measured value of a fluorescence reaction using a probe that reacts with a specific base sequence.

According to a thirteenth aspect of an invention, in the allele determining device according to the twelfth aspect, the fluorescence reaction is an Invader (registered trademark) method.

A fourteenth aspect of an invention provides an allele determining method for determining a single nucleotide polymorphism of a gene, including an approximating step of approximating an optical measurement result obtained by observing a reagent that reacts with a specific base sequence of a gene, to a predetermined curve using light intensity and time as parameters, and a determining step of determining a single nucleotide polymorphism using a characteristic point of the curve that was approximated in the approximating step.

A fifteenth aspect of an invention provides an allele determining method for determining a single nucleotide polymorphism of a gene, including an approximating step of approximating an optical measurement result obtained by observing a reagent that reacts with a specific base sequence of a gene, to a predetermined curve using light intensity and time as parameters, and a determining step of determining a single nucleotide polymorphism using a characteristic point obtained from a logarithm of a curve that was approximated in the approximating step.

A sixteen aspect of an invention provides a computer program, wherein the computer used as an allele determining device that determines a single nucleotide polymorphism of a gene functions as approximating means that approximates an optical measurement result obtained by observing a reagent that reacts with a specific base sequence of a gene, to a predetermined curve using light intensity and time as parameters, and determining means that determines a single nucleotide polymorphism using a characteristic point of the curve that was approximated by the approximating means.

A seventeenth aspect of an invention provides a computer program, wherein the computer used as an allele determining device that determines a single nucleotide polymorphism of a gene functions as approximating means that approximates an optical measurement result obtained by observing a reagent that reacts with a specific base sequence of a gene, to a predetermined curve using light intensity and time as parameters, and determining means that determines a single nucleotide polymorphism using a characteristic point obtained from a logarithm of a curve that was approximated by the approximating means.

### BRIEF DESCRIPTION OF THE DRAWINGS

FIGS. 1A, 1B, 1C and 1D are diagrams showing typical patterns of positive and negative reaction curves;

FIG. 2 is a diagram showing a logistic curve;

FIGS. 3A, 3B and 3C are diagrams showing logistic curves when some of parameters are fixed;

FIG. 4 is a block diagram showing a configuration of an allele determining device according to an embodiment of the present invention;

FIG. 5 is an explanatory diagram of endpoint time of an improved end point algorithm;

FIG. 6 is a diagram showing a probability distribution of ideal fluorescence intensity;

FIG. 7 is a diagram showing a probability distribution of fluorescence intensity when adjustment is insufficient;

FIGS. 8A and 8B are diagrams showing determination examples using corrected data;

FIGS. 9A and 9B are diagrams showing models of transition of index S(t);

FIGS. 10A, 10B, 10C, 10D, 10E and 10F are diagrams showing examples of observation results of RED/FAM;

FIG. 11 is a diagram showing transition of the index S(t) by the observation results in FIG. 10;

FIGS. 12A and 12B are diagrams showing examples of positive and negative inflection points;

FIG. 13 is a diagram showing a relation between an FAM inflection point and a RED inflection point;

FIG. 14 is a diagram showing an example of a case where the FAM inflection point and the RED inflection point are close to each other;

FIGS. 15A and 15B are a flowchart of a general outline of SNP determining processing in the allele determining device;

FIG. 16 is a diagram showing logic of the SNP determining processing in the allele determining device;

FIGS. 17A, 17B and 17C area flowchart of positive/negative determining processing in the allele determining device;

FIGS. 18A and 18B are a flowchart of positive/negative determining processing in the allele determining device;

FIG. 19 is an output processing flowchart of a parameter a in the allele determining device;

FIGS. 20A and 20B are an outputting processing flowchart of an inflection point T1 in the allele determining device;

FIG. 21 is an outputting processing flowchart of a parameter b in the allele determining device;

FIG. 22 is a calculating processing flowchart of a ratio T′ between the FAM inflection point and the RED inflection point in the allele determining device;

FIG. 23 is a diagram showing determination of an end point method applied to an experiment;

FIG. 24 is a plot diagram of a fluorescence value of the end point method obtained by a result of the experiment;

FIG. 25 is a correlation diagram of inflection points T^{F} and T^{R} of a logistic algorithm obtained by the result of the experiment;

FIG. 26 is an explanatory diagram of a measuring method by an Invader (registered trademark) method;

FIGS. 27A, 27B, 27C and 27D are diagrams showing examples of a result pattern of analysis data;

FIGS. 28A, 28B and 28C are explanatory diagrams of the endpoint method;

FIGS. 29A and 29B are explanatory diagrams of rise in background; and

FIG. 30 is a diagram showing time-variation in a fluorescence intensity ratio of FAM and RED.

### DESCRIPTION OF REFERENCE NUMERALS

1 . . . allele determining device, 2 . . . measuring unit (measuring means), 3 . . . storing unit (storing means), 4 . . . approximating unit (approximating means), 5 . . . determining unit (determining means), 6 . . . outputting unit (outputting means)

### DETAILED DESCRIPTION OF THE INVENTION

An embodiment of the present invention will be described with reference to the drawings.

[1. Summary]

An allele determining device according to an embodiment of the present invention determines a genetic pattern of SNP based on a result of measurement optically obtained from a probe that identifies a specific base sequence. The Invader (registered trademark) method, the TaqMan method, the SNaPshot (registered trademark) method, the Sniper method and the like can be used for this technique. Here, the embodiment will be described based on the Invader (registered trademark) method.

The allele determining device of the embodiment brings measured data of light-emission with time by FAM/RED obtained by observation of a sample by the Invader (registered trademark) closely analogous to a predetermined curve, a characteristic point or a coefficient obtained from an equation indicating that curve is analyzed and the SNP is determined based on a result of analysis. Here, a case where a logistic curve is used as a curve to be brought closely analogous is described. For determining the SNP, an inflection point T (corresponding to rising time of curve) is mainly used as the characteristic point of a logistic curve.

[1.1 Characteristic of Reaction Curve]

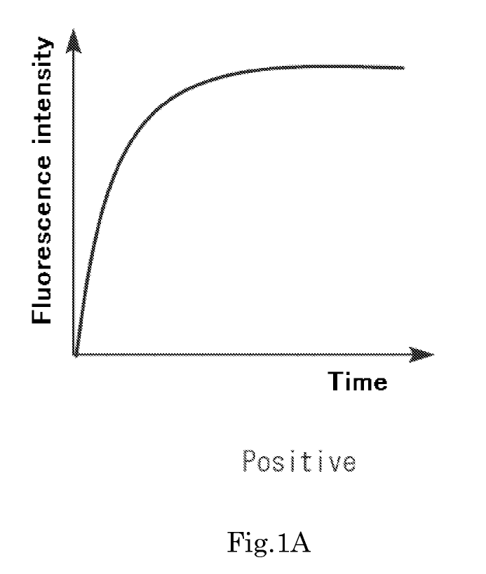

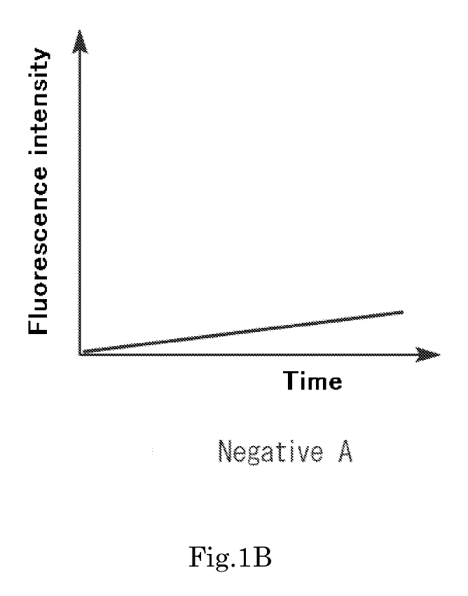

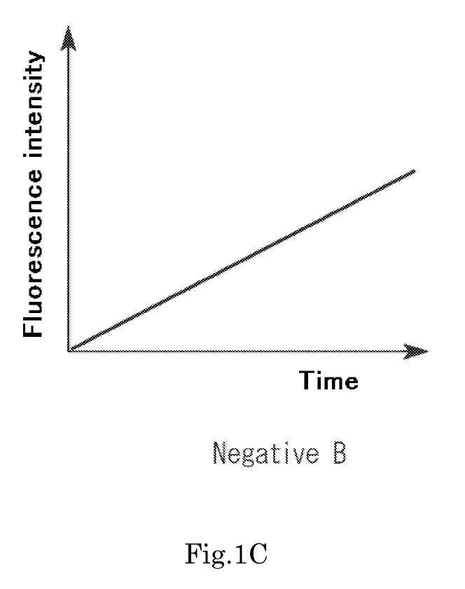

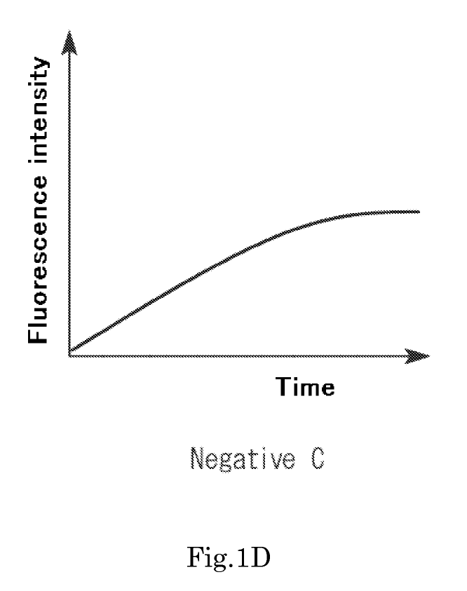

First, characteristics of positive and negative reaction curves will be described. FIG. 1 shows typical positive and negative patterns of the reaction curves indicating fluorescence intensities with time.

As shown in FIG. 1A, a positive reaction curve has the following characteristics.
(1) A plateau value (value when variation disappeared) takes a value equal to or greater than a certain value.
(2) There is a point at which a fluorescence measurement curve abruptly rises.
(3) Rising time is relatively short.

A negative reaction curve has the following characteristics.
(1) There is no point at which a curve abruptly rises, there is no plateau value (value when variation disappeared), and a value close to 0 levels off (FIG. 1B).
(2) Background rises. In this case, some data keeps rising gradually in some cases (FIG. 1C), or some data reaches a plateau (region where a value is not varied) (FIG. 1D). However, even a reaction curve that seems to reach the plateau, the plateau value is low and rising time is relatively late.

Specifically, a parameter of a logistic curve shows a plateau value in reaction, rising time and the like. Hence, it is possible to largely reduce erroneous determination by applying an algorithm using a parameter that is actually obtained by approximation to a logistic curve to the SNP determination. This is because that erroneous determination is made by the end point method concerning data having a vague fluorescence value or negative data in which end point rises, but if an inflection point of a logistic curve is used as an index, it can be rejected. Therefore, it is possible to realize an allele determining device having less erroneous determination by using this embodiment.

[1.2 Characteristic of Logistic Curve]

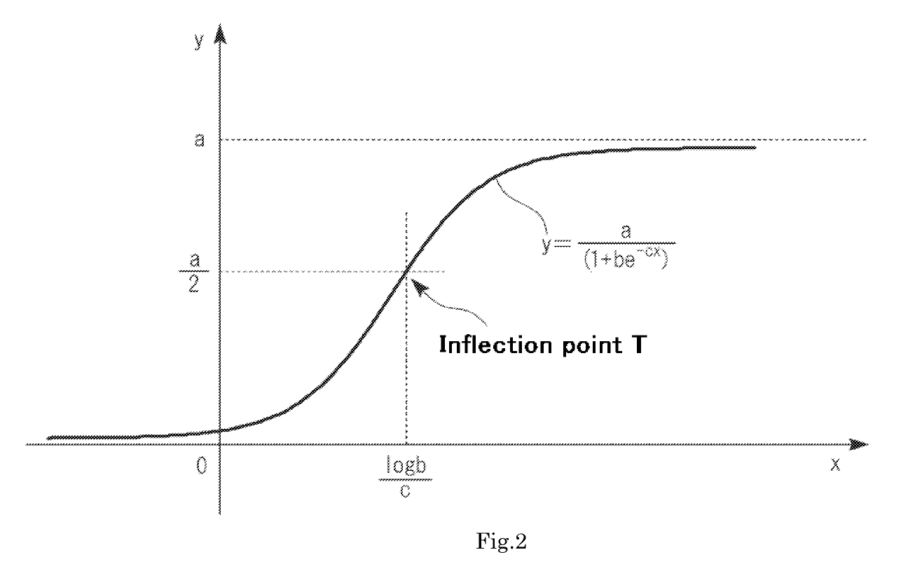

FIG. 2 is a diagram showing a logistic curve.

The logistic curve is a curve in which a growth of an organism (e.g., population growth) is modeled, and is frequently utilized as a typical pattern of an S-shaped curve or a sigmoid curve. A model of the logistic curve is expressed as in the following equation, wherein a represents an index of maximum value (maximum value of logistic curve approaches a), b represents parallel movement of horizontal axis, and c represents a rising speed.

y=a/(1+be^{-cx})

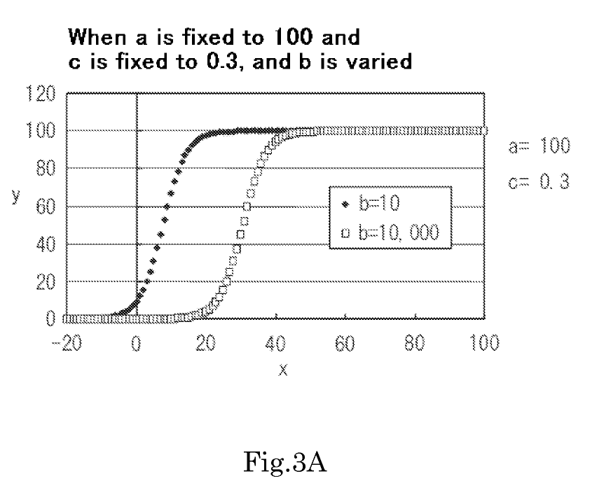

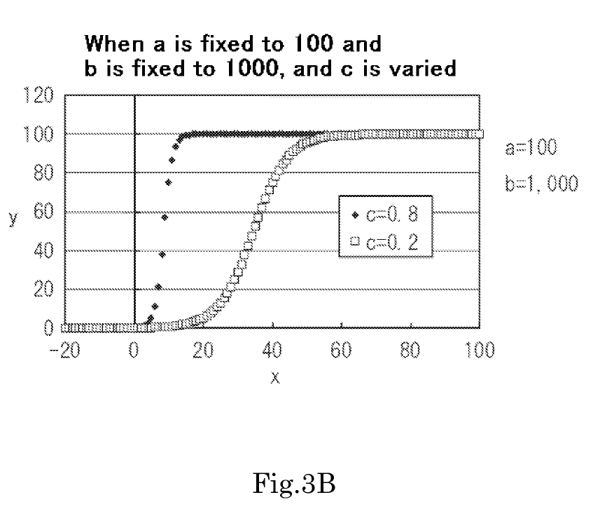

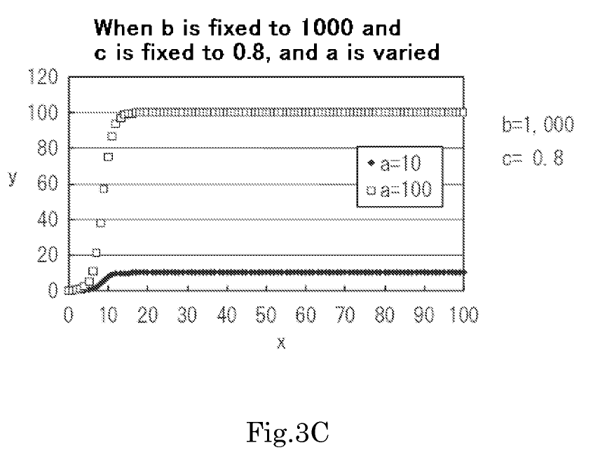

The logistic curve takes inflection point (x, y)=((logb)/c, a/2), and is a curve that is symmetric with respect to the inflection point. FIG. 3A shows a logistic curve when a is fixed to 100, c is fixed to 0.3 and b is varied. FIG. 3B shows a logistic curve when a is fixed to 100, b is fixed to 1000 and c is varied. FIG. 3C shows a logistic curve when b is fixed to 1000, c is fixed to 0.8 and a is varied.

[1.3 Measurement for Solving Problem]

To solve a problem caused when the conventional endpoint method is used, countermeasure that is realized by the allele determining device of the embodiment will be described.

(Countermeasure 1) Processing Using Time-Varying Data (Real-Time Processing);

In the end point method, measurement is performed with fixed timing (after two minutes in a conventional standard protocol), but it is possible to measure at the optimal timing by following the time-varying data. It is possible to compare not only an intensity ratio but also rising speeds in a reaction curve. When real-time processing is difficult, it is also possible to analyze time-varying data of a plurality of standard samples by a later-described method, and to adjust the optimal measuring time.

(Countermeasure 2) Use of Positive Control (Or Standard Sample Data);

To match intensity ratios (scales) of FAM and RED, positive control data or standard sample data is utilized instead of utilizing conventional negative control data.

(Countermeasure 3) Approximation of Data (Application of Logistic Curve);

By bringing the entire time-varying data closely analogous to a logistic curve, it is possible to reduce influence of noise and simple calculation is realized.

(Countermeasure 4) Improvement of Calculation Method of Ratio (Solve Asymmetry);

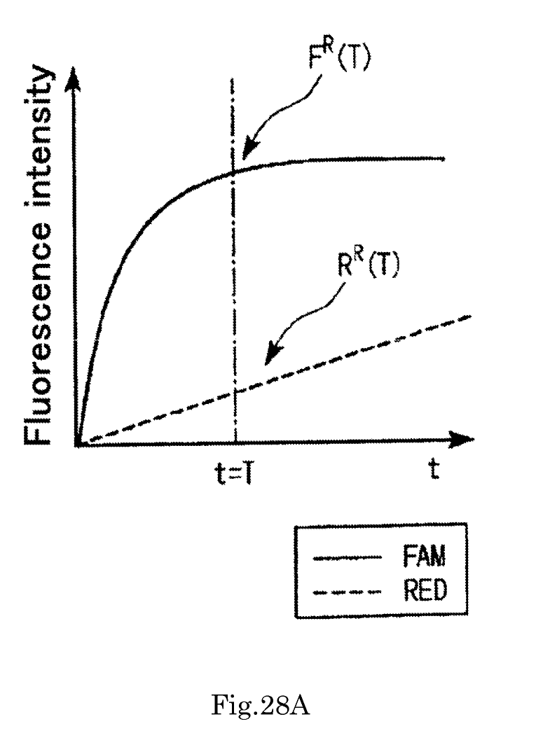

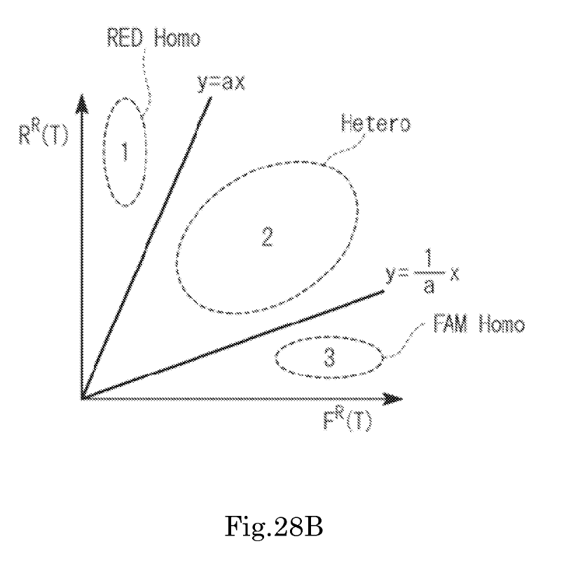

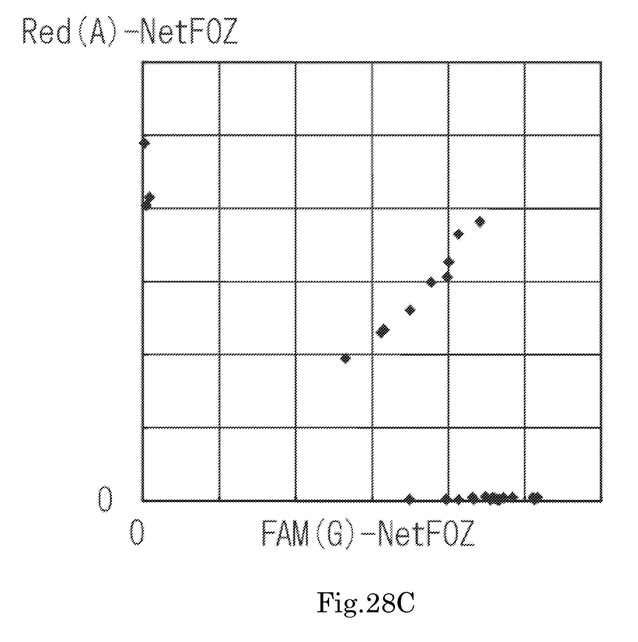

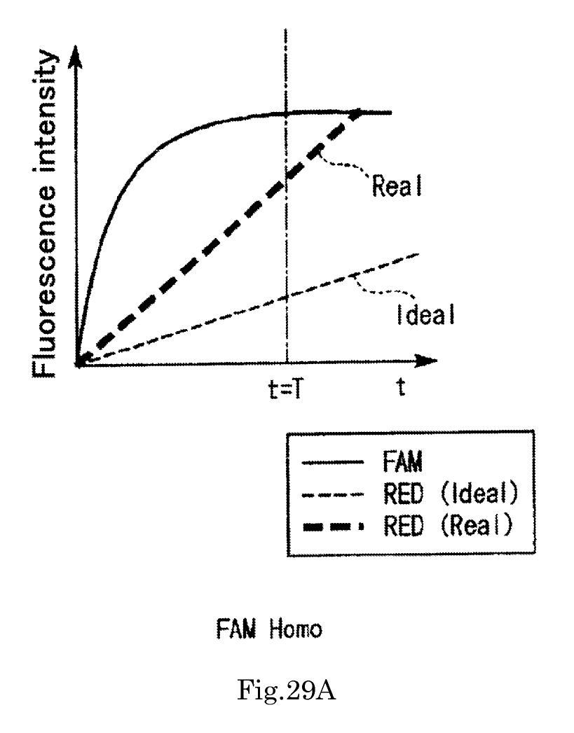

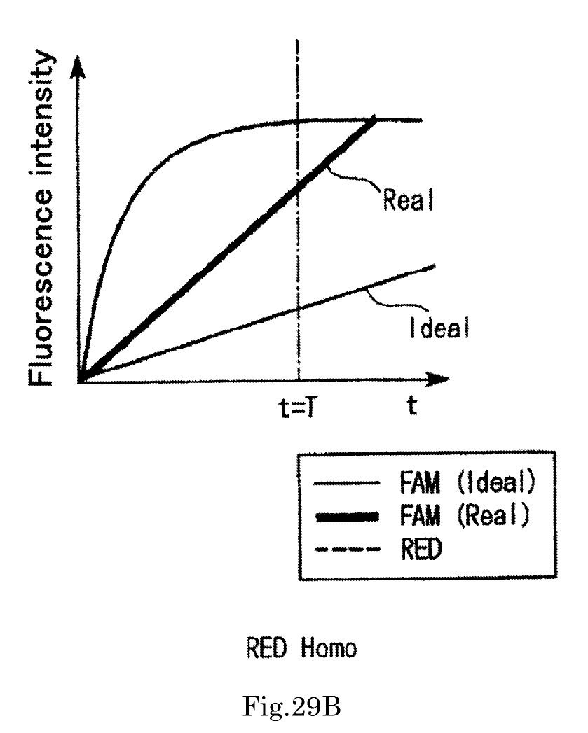

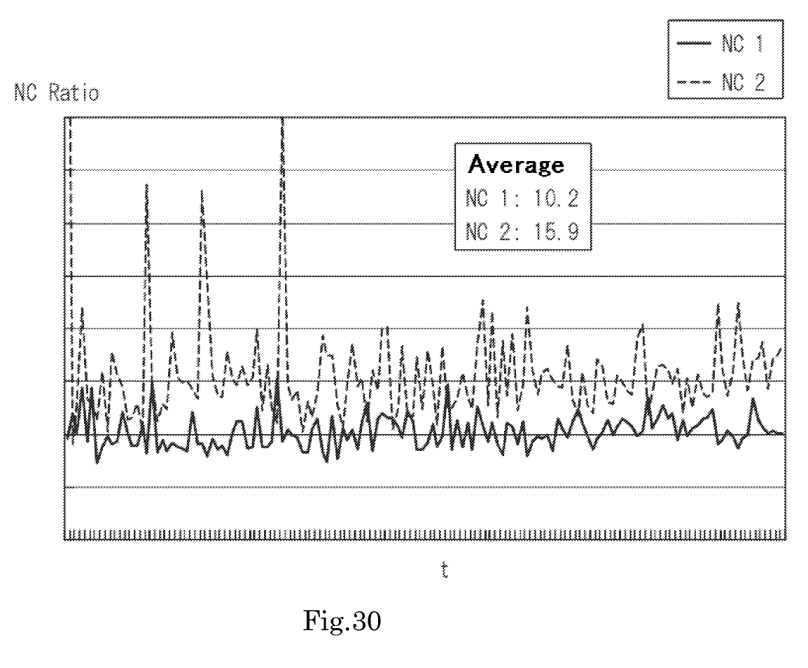

Conventionally, concerning a value of Ratio=F^{R}/R^{R}, determination was made using straight lines in which inclinations are a and 1/a were boundaries as parameters (see FIG. 28B). Hence, to provide the two parameters with symmetry, logarithm logs are utilized as follows.

log (Ratio)=log (F^{R}/R^{R})=log (F^{R})−log (R^{R})

With this, RED Homo can be determined as log (Ratio)<-loga, Hetero can be determined as -loga<log (Ratio)<loga, and FAM Homo can be determined as log(Ratio)>loga.

[2. Configuration of Allele Determining Device, and Packing Algorithm] [2.1 Configuration of Device]

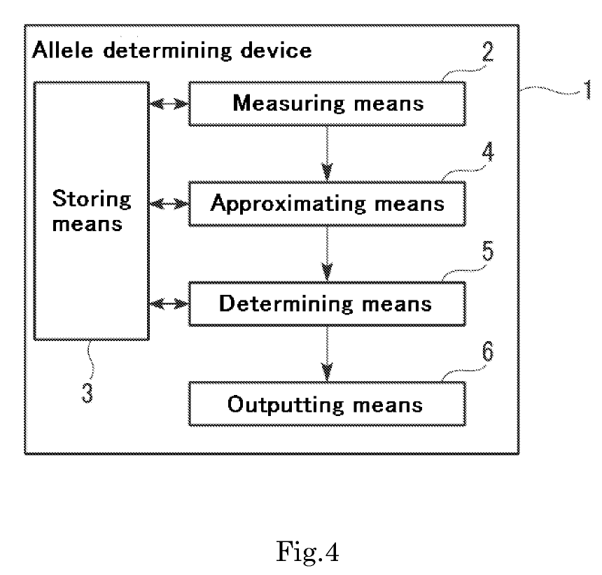

FIG. 4 is a block diagram showing a configuration of the allele determining device 1 according to an embodiment of the present invention in which only function blocks related to the invention are extracted.

The allele determining device 1 includes a measuring unit 2, a storing unit 3, an approximating unit 4, a determining unit 5 and an outputting unit 6. The measuring unit 2 performs optical measurement, and obtains fluorescence intensities of FAM and RED by the Invader (registered trademark) method. The storing unit 3 stores therein various data sets of fluorescence intensities measured by the measuring unit 2 and various data sets used for determining processing. The approximating unit 4 brings a reaction curve closely analogous to a predetermined curve, here a logistic curve in which intensity of light and time are used as parameters from a result of measurement of intensity of fluorescence obtained by the measuring unit 2. The determining unit 5 determines single nucleotide polymorphism using a characteristic point of a curve approximated by the approximating unit 4. The outputting unit 6 shows a result of determination made by the determining unit 5 on a display, or writes the same in a storing medium, or sends the same to a computer terminal connected through a network.

[2.2 Determining Algorithm] [2.2.1 Logistic Algorithm]

Algorithm that is executed by the allele determining device 1 of the embodiment based on the above-described countermeasures will be described. Here, there will be described a logistic algorithm in which a fluorescence value indicated by observed value data is non-curve regression analyzed to bring it closely analogous to the logistic curve, and SNP is determined using a parameter of the obtained approximation curve equation. In the following description, (t) represents a value at time t elapsed after measurement is started.

(Procedure 1) The measuring unit 2 obtains the time-series data, i.e., sample data (raw data), negative control (NC) data, positive control (PC) data as an observation result, and writes them in the storing unit 3.

The sample data (raw data) is FAM fluorescence intensity F^{A}(t) and RED fluorescence intensity R^{A}(t) of samples.

The negative control (NC) data is FAM fluorescence intensity F^{N}(t) and RED fluorescence intensity R^{N}(t) of negative control. This is negative control data measured without sample, and the same elements were measured for the reagents other than the samples.

The positive control (PC) data is FAM fluorescence intensity F^{P}(t) and RED fluorescence intensity R^{P}(t) of positive control. These are positive control data measured with standard samples, and this measured value is a reference of normal reaction.

(Procedure 2) The approximating unit 4 corrects data of negative control as follows for sample data and positive control data obtained in (procedure 1).

Sample Data:

F^{AR}(t)=F^{A}(t)−FN(t),

R^{AR}(t)=R^{A}(t)−RN(t),

wherein, if F^{AR}(t)<0, F^{AR}(t)=0.

Positive Control Data:

F^{PR}(t)=F^{P}(t)−F^{N}(t),

R^{PR}(t)=R^{P}(t)−R^{N}(t),

wherein, if F^{PR}(t)<0, F^{PR}(t)=0.

(Procedure 3) The approximating unit 4 approximates logistic curve y=a/(1+be^{-cx}) by the method of least squares or the like using positive control data F^{PR}(t) and R^{PR}(t) obtained in the (procedure 2), and obtains parameters a, b and c.

Positive control FAM: the parameters a, b and c are calculated using positive control data F^{PR}(t), and the answers are defined as a^{PF}, b^{PF} and c^{PF}.

Positive control RED: the parameters a, b and c are calculated using positive control data R^{PR}(t), and the answers are defined as a^{PR}, b^{PR} and c^{PR}.

(procedure 4) The approximating unit 4 carries out the following calculations for the positive control parameters obtained in the (procedure 3).

Positive control FAM: p^{F}=a^{PF}

Positive control RED: p^{R}=a^{PR}

(Procedure 5) The approximating unit 4 approximates logistic curve y=a/(1+be^{-cx}) by the method of least squares or the like using sample data F^{AR}(t) and R^{AR}(t) obtained in the (procedure 2) and obtains parameters a, b and c.

Sample FAM: parameters a, b and c are calculated using sample data F^{AR}(t), and the answers are defined as a^{AF}, b^{AF} and c^{AF}, respectively.

Sample RED: parameters a, b and c are calculated using sample data R^{AR}(t), and the answers are defined as a^{AR}, b^{AR} and c^{AR}, respectively.

(Procedure 6) The approximating unit 4 calculates the following equations as corrected data.

$$Corrected data \text{FAM} \text{:} \\ {F}^{R} ( t ) = \frac{( \frac{{a}^{AF}}{1 + {b}^{AF} {e}^{- {c}^{AF} t}} )}{{p}^{F}} \\ Corrected data \text{RED:} \\ {R}^{R} ( t ) = \frac{( \frac{{a}^{AR}}{1 + {b}^{AR} {e}^{- {c}^{AR} t}} )}{{p}^{R}} \qquad [ Equation 1 ]$$

(Procedure 7) The approximating unit 4 calculates the inflection point.

Inflection point of FAM: T^{F}=(logb^{AF})/c^{AF}

Inflection point of RED: T^{R}=(logb^{AR})/c^{AR}

(Procedure 8) The determining unit 5 makes a determination utilizing inflection points T^{F} and T^{R} (collectively called “inflection point T”, hereinafter) calculated in (procedure 7).

The following index is utilized for an approximated curve.

S(t)=log(F^{R}(t)/R^{R}(t))

[2.2.2 Improved End Point Algorithm]

Next, an algorithm that is executed by the allele determining device 1 and in which end point method is improved will be described.

(Procedure 1) The measuring unit 2 measures a standard sample in real time and writes the measured data into the storing unit 3. The number of necessary samples depends on deviation of the measured values.

(Procedure 2) The approximating unit 4 approximates to a logistic curve in accordance with the logistic algorithm described in the paragraph 2.2.1, and estimates each parameter and checks a variation degree thereof.

(Procedure 3) The determining unit 5 calculates the optimal measuring time T′.

(Procedure 4) The determining unit 5 makes the following determination using the optimal measuring time T′ as measuring time of the end point.

If a reaching degree of a plateau a is formulated with α, time at which a measured value reaches a(1−α) is expressed in the following equation. Here, T=(logb)/c is time of an inflection point.

$${T}^{′} = \frac{log b - log ( \frac{α}{1 - α} )}{c} = T - \frac{1}{c} log ( \frac{α}{1 - α} ) \qquad [ Equation 2 ]$$

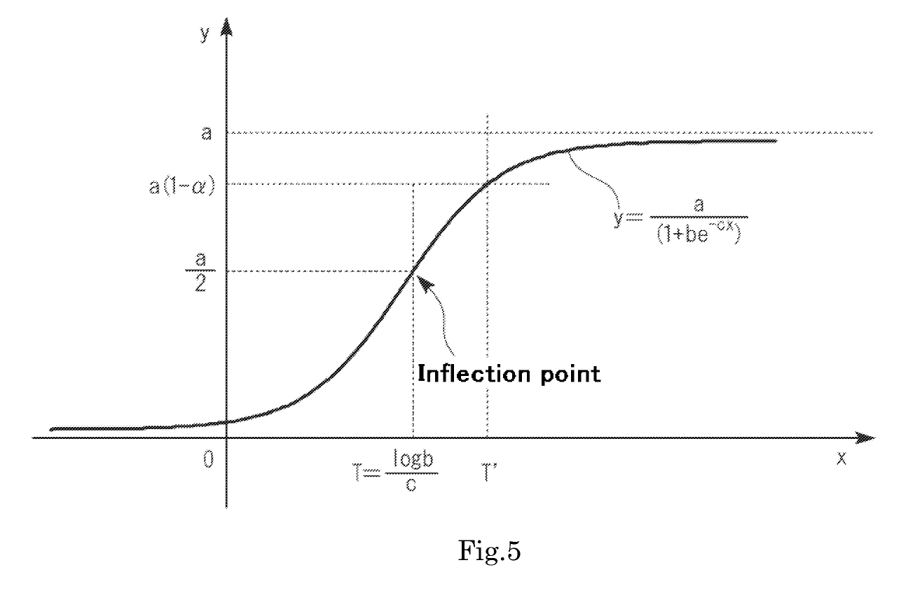

For example, when a plateau is 97%, α becomes equal to 0.03. When b=200 and c=0.1 and α=0.03, T=17. 7, T′=27.6 and T′/T=1.56. This means that if time of 1.56 times of time of the inflection point is measured, a value of 97% of a plateau can be measured. This is shown in FIG. 5.

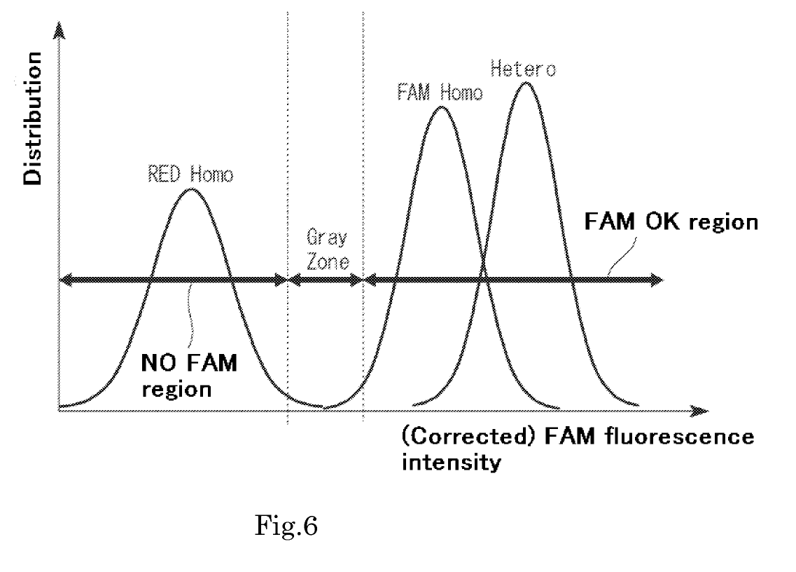

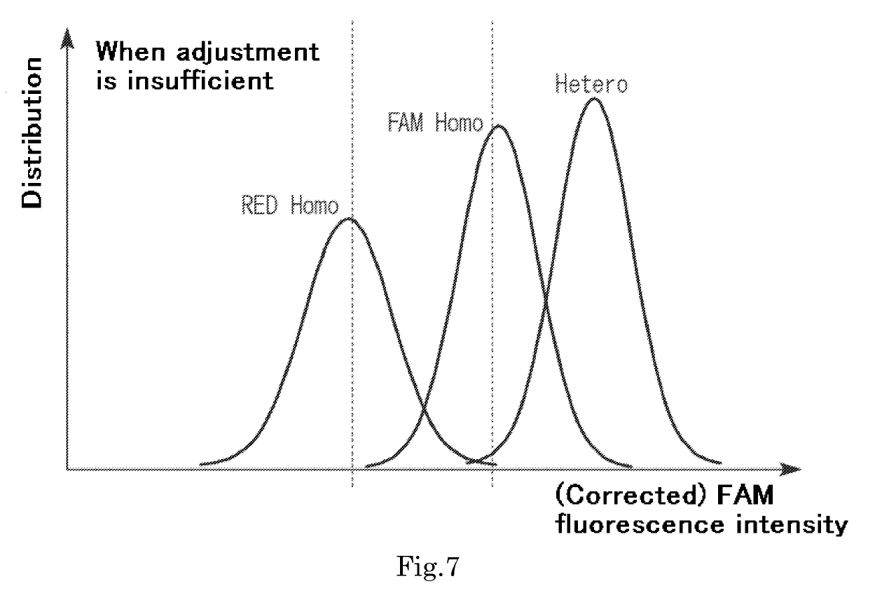

An actual measured value includes various errors. It is necessary to determine a probability distribution of errors by checking the actual measured values. If parameters of a, b and c obey probability distributions that are independent from each other, it is estimated that a measured value of an end point of time draws a probability distribution as shown in FIG. 6. In the drawing, probability distributions of fluorescence intensities of the RED Homo, and of FAM Homo and Hetero are separated from each other, and a grey zone that is an intensity range where FAM fluorescence intensity is not observed is clear. When the end point is measured, it is necessary to analyze the actual data to determine whether the determination should be made simply in a region where a value exists or a FAM/RED ratio should be employed. It is necessary to adjust conditions of reagents and temperature, and to clearly separate a RED Homo region where FAM is not observed and a FAM Homo and Hetero region where FAM is observed from each other. When the adjustment is insufficient, probability distributions of fluorescence intensities of the RED Homo and the FAM Homo are separated from each other as shown in FIG. 7, and a fluorescence intensity range where both the RED Homo and FAM of the FAM Homo can be observed adversely appears.

[2.3 Example of Determination]

The SNP determining processing using the algorithm that is executed in the determining unit 5 of the allele determining device is shown below.

[2.3.1 Example of Determination Using Corrected Data F^{R}(t) and R^{R}(t)]

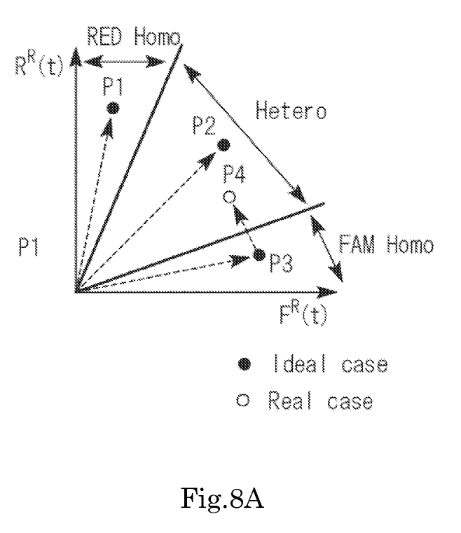

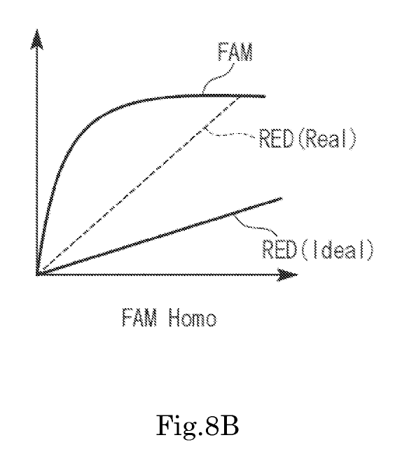

In FIG. 8A, P1, P2 and P3 show transition of corrected data F^{R}(t) and R^{R}(t) for the RED Homo, Hetero and FAM Homo in an ideal sates are plotted as P1, P2 and P3. These P1, P2 and P3 are plotted in the RED Homo region, the Hetero region and the FAM Homo region divided by two straight lines.

The allele determining device 1 previously stores, in the storing unit 3, information of straight lines for dividing a plane having two axes of the corrected data F^{R}(t) and R^{R}(t) into the RED Homo region, the Hetero region and the FAM Homo region, the determining unit 5 determines to which region the corrected data F^{R}(t) and R^{R}(t) calculated in the procedure 6 of the logistic algorithm shown in 2.2.1 belongs using the stored information, and determines one of the RED Homo, Hetero and FAM Homo depending the region to which it belongs.

However, when background rises as shown with a dotted line in FIG. 8B, data that should originally enter the FAM Homo enters the Hetero region as P4 plot in FIG. 8A, and this may cause a erroneous decision.

[2.3.2 Example of Determination Using Index S(t)]

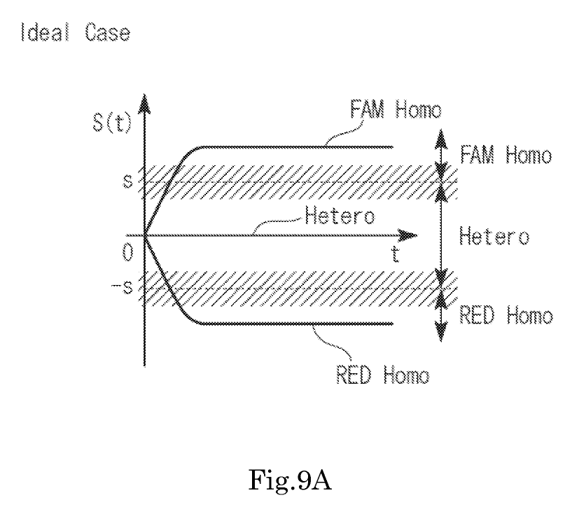

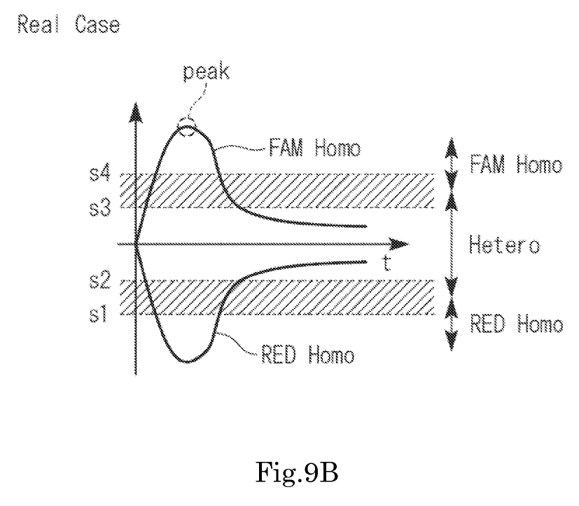

Transition of the corrected data F^{R}(t) and R^{R}(t) is conceived using index S (t)=log (F^{R}(t)/R^{R}(t)) calculated by the (procedure 8) of the logistic algorithm shown in 2.2.1. If the transition S (t) is represented in graphic form, it becomes as shown in FIG. 9B in its ideal state. According to this drawing, if S (t)>s when the value S (t) stabilizes (variation disappears), it can be determined to be FAM Homo, if −s<S (t)<s, it can be determined to be Hetero, and if S (t)<−s, it can be determined to be RED Homo.

However, if background rises actually, the transition of S (t) becomes as shown in FIG. 9B. That is, if determination is made when the S (t) value stabilizes (variation disappears), time elapses and the FAM Homo and RED Homo enters the Hetero area (quite the same as the case shown in FIG. 8). Hence, it is conceived that determination is made around a peak value or its periphery where the S (t) value becomes maximum or minimum. That is, a speed ratio S (t) when a difference between rising speeds of FAM and RED becomes the maximum is seen. Specifically, in FIG. 9B, grey zones are taken into consideration, the determining unit 5 of the allele determining device 1 determines to be FAM Homo if s4<Peak value, and determines to be Hetero if s2<Peak value<S3, and determines to be RED Homo if Peak value<s1. Values of s1, s2, s3 and s4 are previously stored in the storing unit 3.

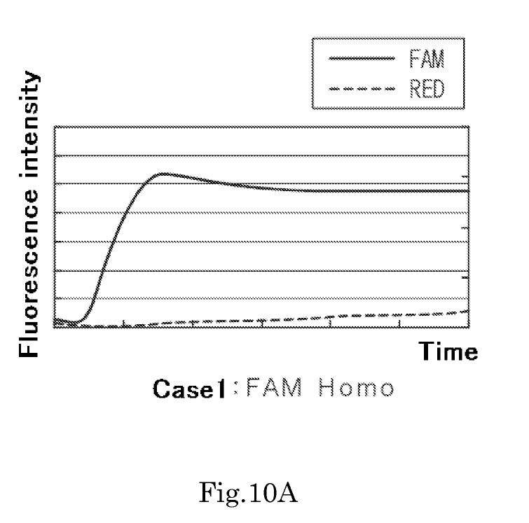

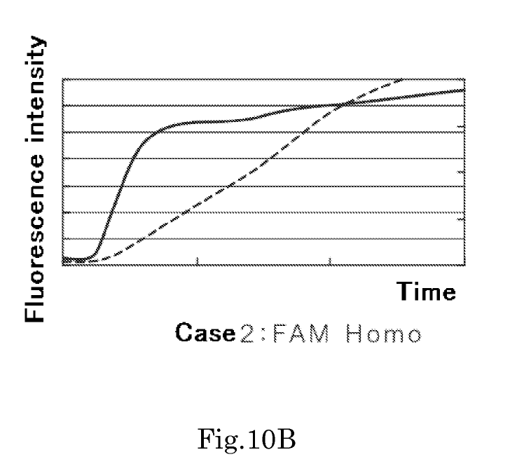

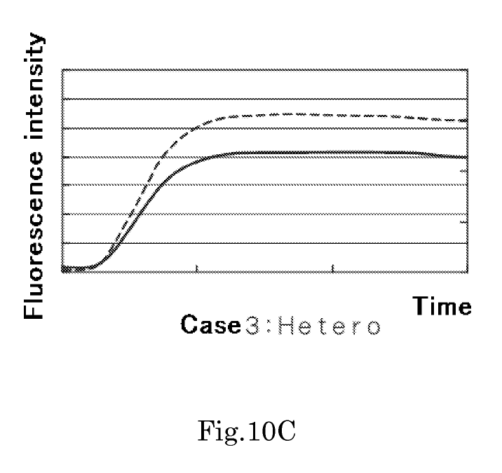

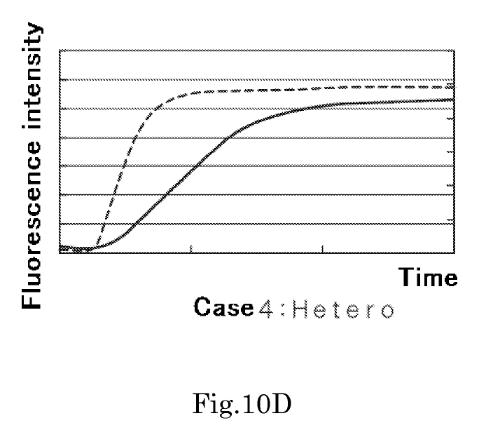

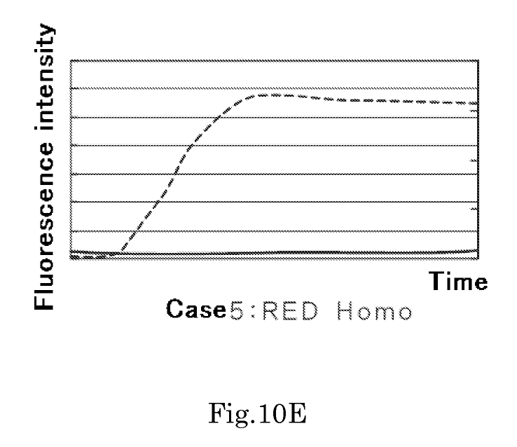

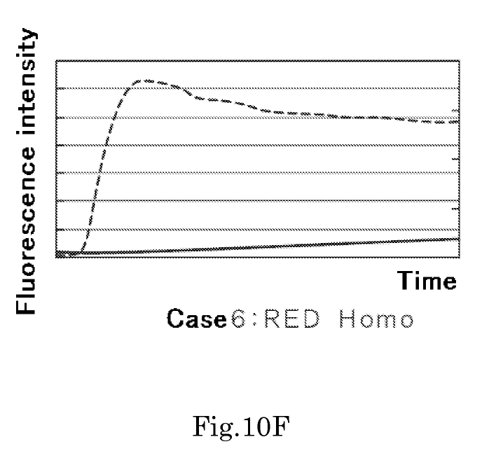

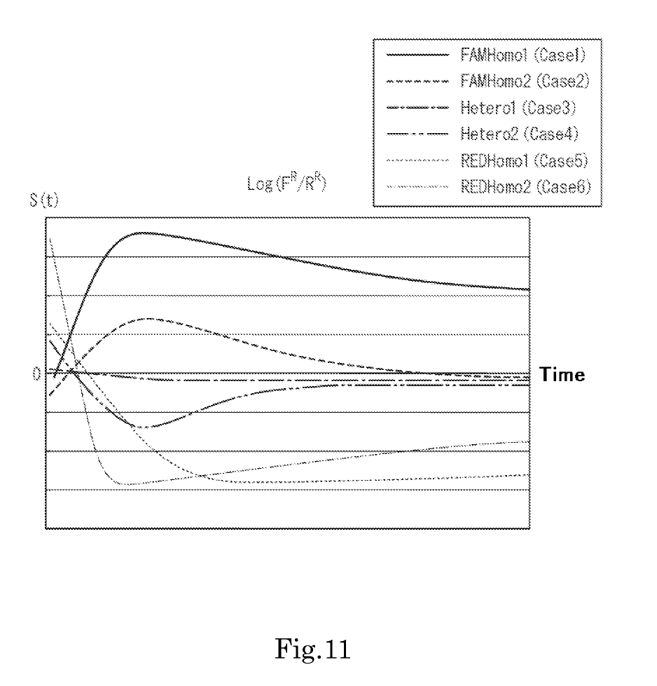

FIG. 10 shows examples of observation results of RED/FAM. FIG. 10A shows an observation result of FAM Homo of a case 1. FIG. 10B shows an observation result of FAM Homo of a case 2. FIG. 10C shows an observation result of Hetero of a case 3. FIG. 10D shows an observation result of Hetero of a case 4. FIG. 10E shows an observation result of RED Homo of a case 5. FIG. 1OF shows an observation result of RED Homo of a case 6. FIG. 11 shows variation with time of S (t) of FIGS. 10A to F. In FIG. 11, peak values of S (t) are great positive values in cases 1 and 2 in the case of FAM Homo, and peak values of S (t) are great negative values in cases 5 and 6 in the case of RED Homo.

In the case of Hetero of case 3, S (t) is close to 0. In the case of Hetero of case 4, peak is seen on the negative side, but its absolute value is small.

[2.3.3 Example of Determination Utilizing Inflection Points T^{F} and T^{R}]

Specific examples of determination utilizing inflection points T (T^{F}, T^{R}) calculated in the (procedure 7) and (procedure 8) of logistic algorithm are shown below.

An inflection point I^{F} of FAM is calculated by T^{F}=(logb^{AF})/c^{AF}, and an inflection point T^{R} of RED is calculated by T^{R}=(logb^{AR})/c^{A} based on parameters obtained from the approximated logistic curve. These inflection points T^{F} and T^{R} correspond to rising time of the fluorescence intensity.

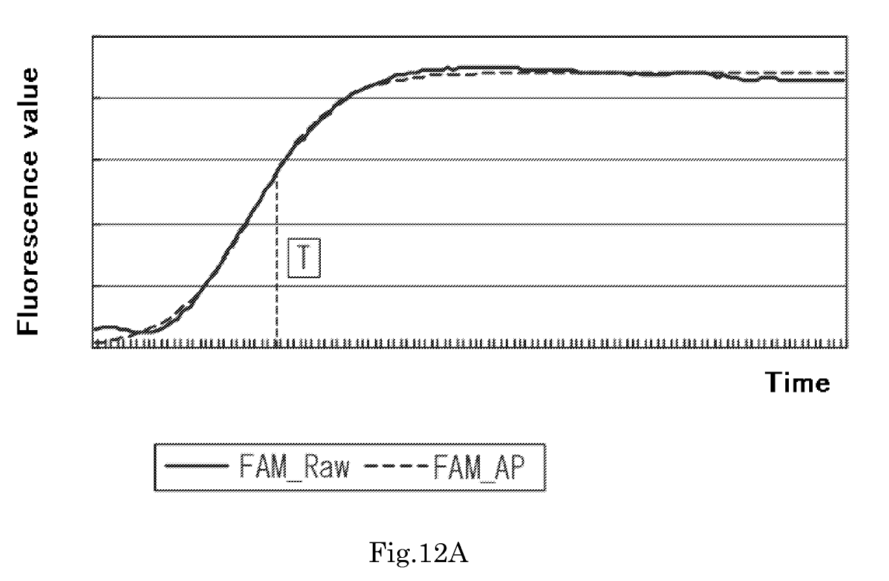

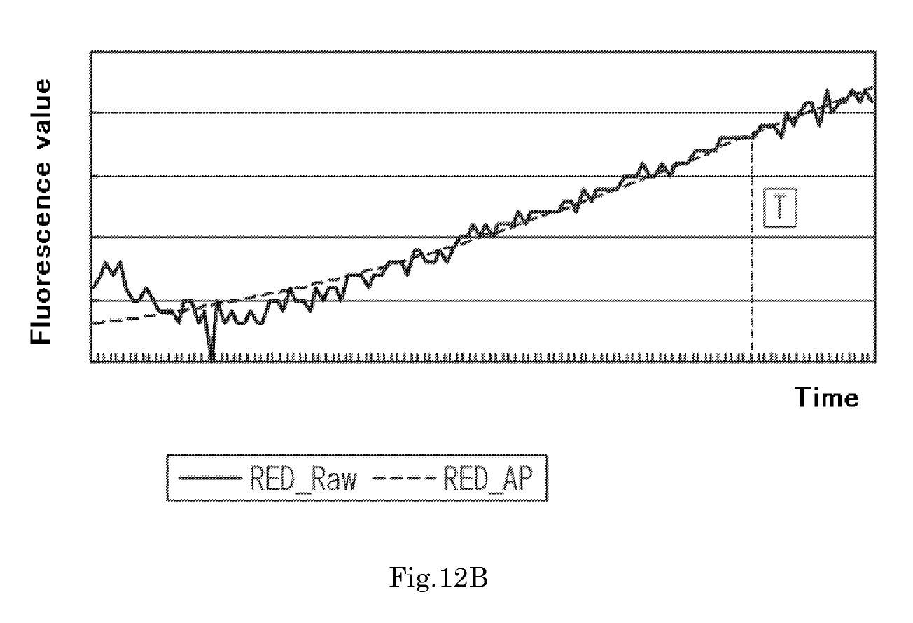

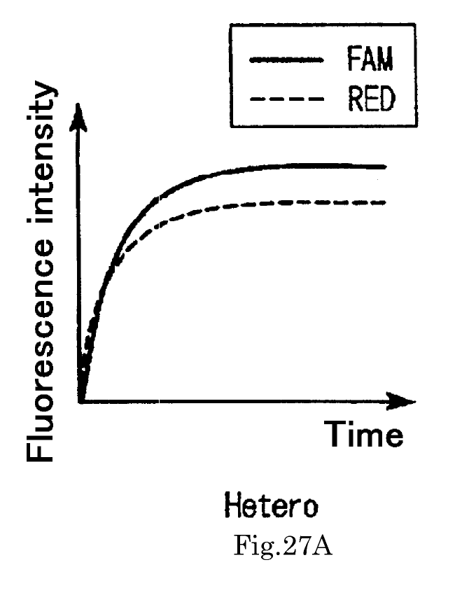

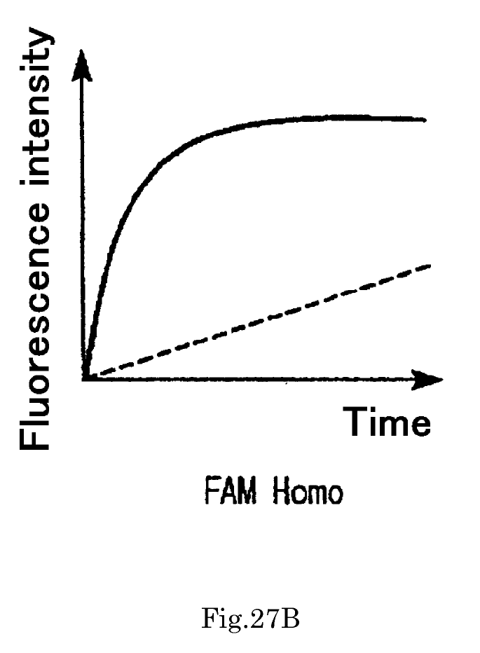

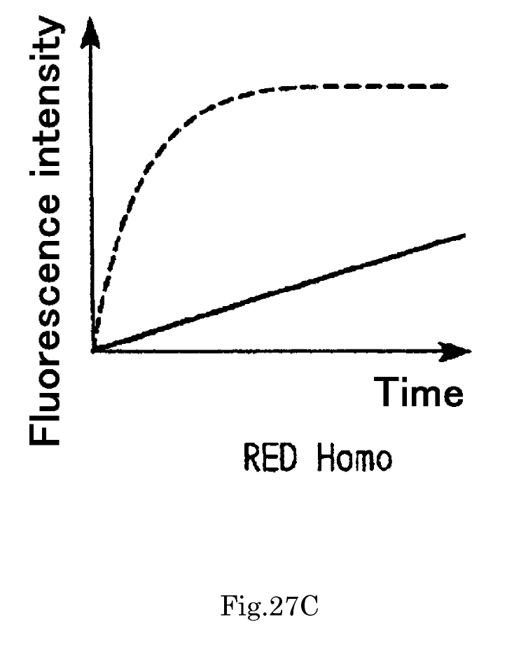

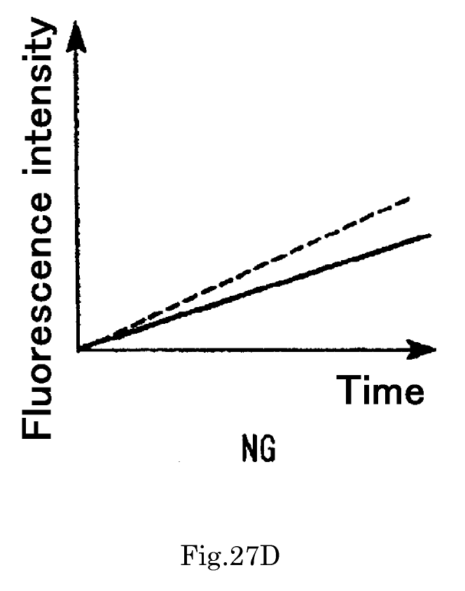

FIG. 12 shows examples of the inflection points T^{F} and T^{R} when measured data of actual FAM and RED is brought closely analogous to logistic curves. Here, observed data of FAM Homo as shown in FIG. 27B is applied to the logistic curve, FIG. 12A shows observed data of FAM and approximated logistic curve, and FIG. 12B shows observed data of RED and approximated logistic curve. Concerning scales of horizontal axes in FIGS. 12A and B with respect to the same scales of vertical axes, A is four times of B. As shown in the drawings, an inflection point T takes a small value when positive, and takes a large value when negative.

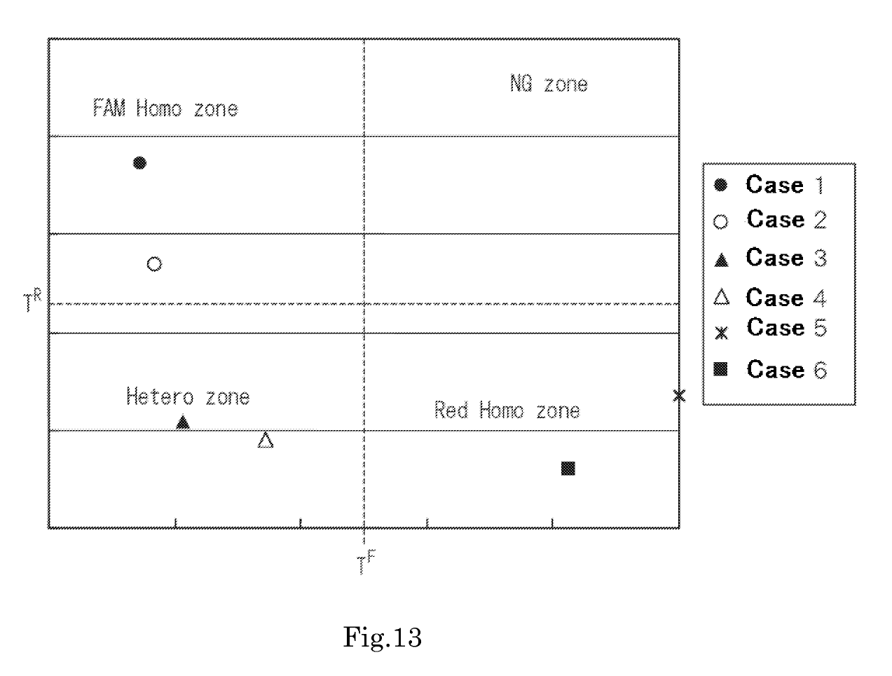

FIG. 13 is a graph in which a horizontal axis shows an inflection point T^{F}=T (FAM) of FAM, and a vertical axis shows an inflection point T^{R}=T (RED) of RED, and they are obtained by plotting the inflection points T^{F} and T^{R} obtained concerning FIGS. 10A to F. As shown in FIG. 13, it is found that concerning FAM Homo, Hetero and RED Homo, four zones divided by dotted lines are clearly divided (when NG region is included).

Hence, the allele determining device 1 previously stores, in the storing unit 3, information of regions to be divided into RED Homo, Hetero, FAM Homo and NG on a plane where the inflection points T^{F} and T^{R} are two axes, the determining unit 5 determines to which region the inflection points T^{F} and T^{R} calculated in the procedure 7 of 2.1.1 belongs using the stored information, and determines one of RED Homo, Hetero, FAM Homo and NG depending upon the region to which the inflection point belongs.

[2.3.4 Determining Method in which Various Indices are Combined in Addition to the Inflection Points T^{F} and T^{R}]

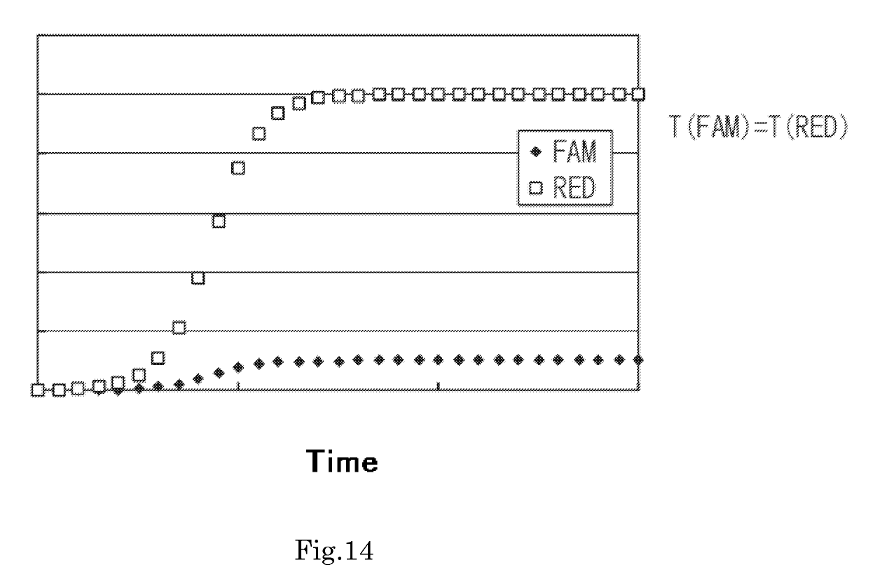

When the region is divided into four, i.e., FAM Homo, Hetero, RED Homo and NG by the method described in 2.3.3, as shown in FIG. 14, it is appeared to be difficult to determine pattern of SNP using only T as an index when T of data that should originally be negative and T of positive data are about the same, or when T of negative data becomes 0. Hence, determination using other index is also executed as shown below.
(1) The inflection point T (T^{F}, T^{R}) corresponds to rising time of sample data. If the rising time of the inflection point T is early, it is determined to be positive, and if late, it is determined to be negative.
(2) The maximum value M^{F} of FAM sample data and the maximum value M^{R} of the RED sample data are utilized. When M^{F} and M^{R} take values equal to or greater than certain values, they become indices of abnormal values.
(3) Parameter a of logistic curves are utilized. When the parameters a (a^{AF}, a^{AR}) of approximated logistic curves of FAM and RED are normal positive data in addition to a case where they become maximum indices, they take values equal to or lower than certain values. Thus, it is compared with a normal maximum value that can be employed for each of the parameter a, and if it is equal to or less than the maximum value, it is determined as being normal.
(4) A ratio T′ of inflection points T of FAM and RED is utilized. When in the case of FAM Homo or RED Homo, the inflection point T (FAM) of FAM and the inflection point T (RED) of RED are largely different from each other as compared with Hetero. Hence, if the ratio T′ is seen, it is possible to determine positive or negative more precisely.

[3. Processing Flow]

Next, processing flow of the allele determining device 1 having the algorithm will be explained.

[3.1 SNP Determining Processing Flow]

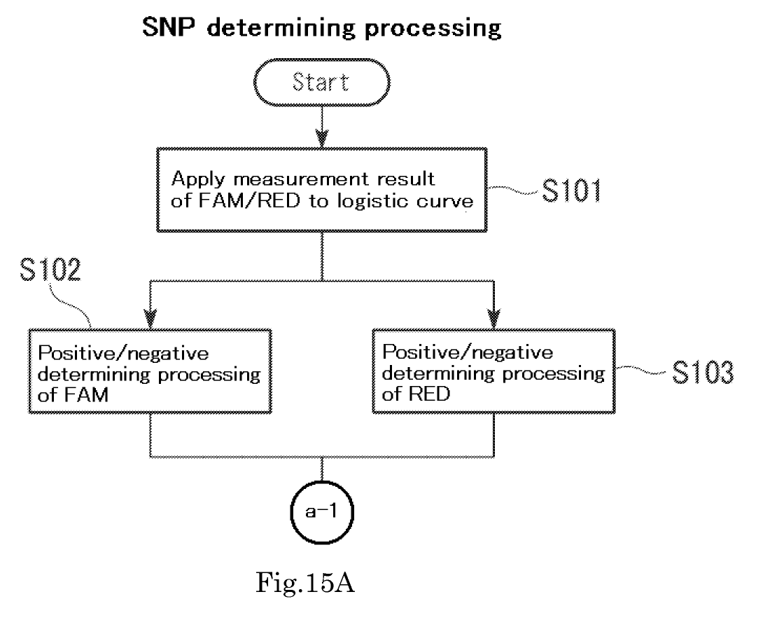

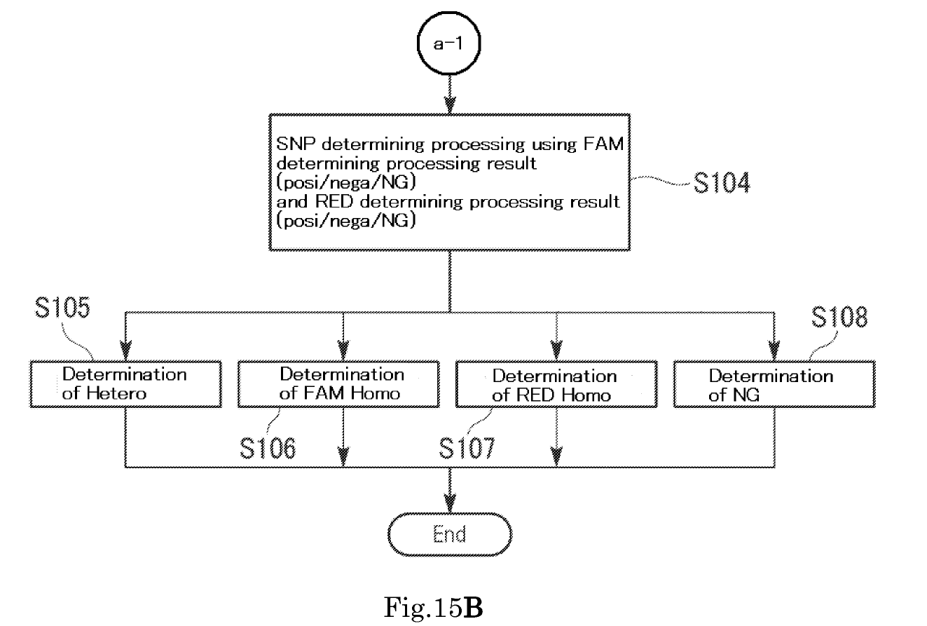

FIG. 15 is a flowchart of an outline of SNP determining processing in the allele determining device 1.

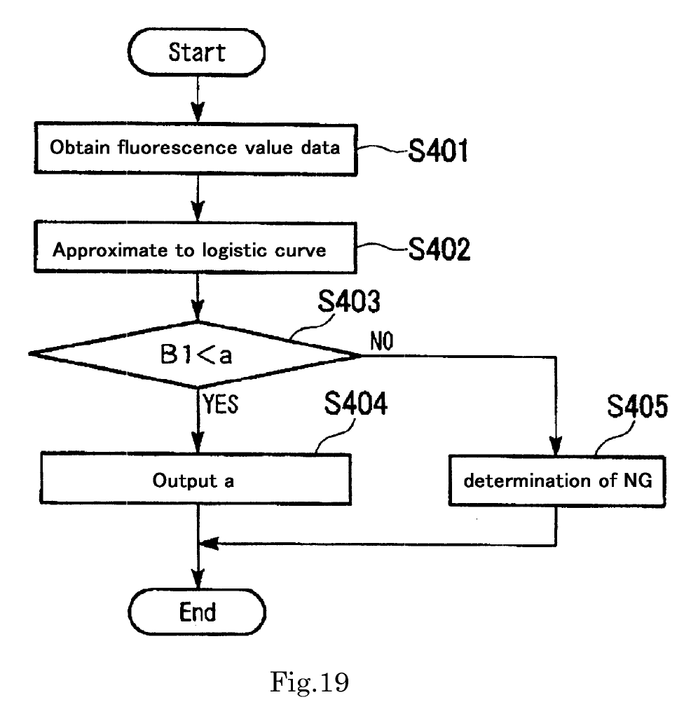

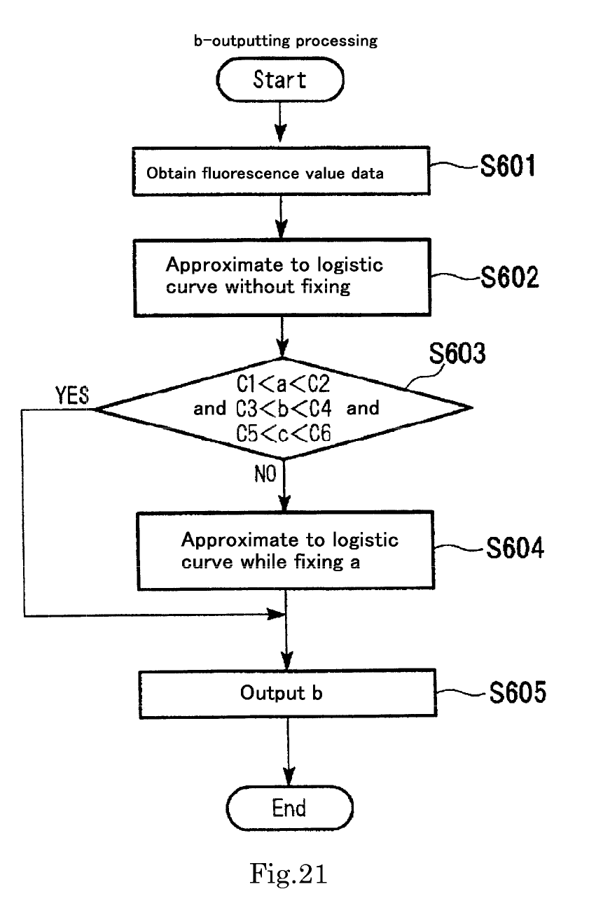

The measuring unit 2 of the allele determining device 1 writes, in the storing unit 3, data of results of measurement obtained by observing FAM fluorescence intensity F^{A}(t) and fluorescence intensity R^{A}(t) of RED of samples of subjects of SNP determination, FAM fluorescence intensity F^{N}(t) of negative control (NC) and fluorescence intensity R^{N}(t) of RED, FAM fluorescence intensity F^{P}(t) of positive control (PC) and RED fluorescence intensity R^{P}(t). The approximating unit 4 reads data of the measurement results from the storing unit 3, executes logistic algorithm described in 2.2.1, approximates FAM and RED to logistic curves, and calculates parameters and indices (step S101). FIGS. 19 and 21 show calculation of the parameters and indices of a case where the negative control (NC) or positive control (PC) is not used.

The determining unit 5 determines that each of FAM and RED is positive (posi), negative (nega) or NG using parameters, indices, measurement result data of the approximate expression of logistic curve obtained for FAM and RED (steps S102, S103). The determining unit 5 of the allele determining device 1 determines SNP based on FAM and RED determining processing result obtained in steps S102, S103 (step S104). The outputting unit 6 obtains a result of the SNP determining processing in step S104 of the determining unit 5, and outputs FAM Homo/Hetero/RED Homo/NG (steps S105 to S108).

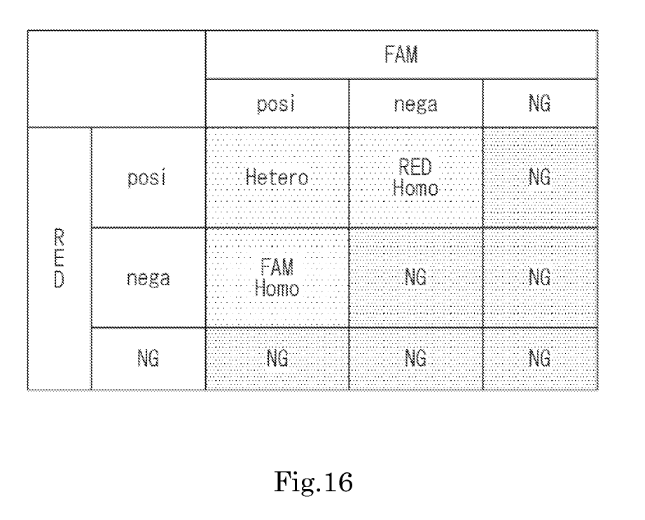

FIG. 16 shows logic of the SNP determining processing in step S104 shown in FIG. 15. When both FAM determination result and RED determination result are positive (posi), the determining unit 5 determines that SNP determination result is Hetero. When FAM determination result is positive (posi) and RED determination result is negative (nega), the determining unit 5 determines that the SNP determination result is FAM Homo, and when RED determination result is positive (posi) and FAM determination result is negative (nega), the determining unit 5 determines that SNP determination result is RED Homo. In other cases, i.e., when any of FAM determination result and RED determination result is NG, or when both FAM determination result and RED determination result are negative (nega), the determining unit 5 determines that SNP determination result is NG.

[3.2 Positive/Negative Determining Flow]

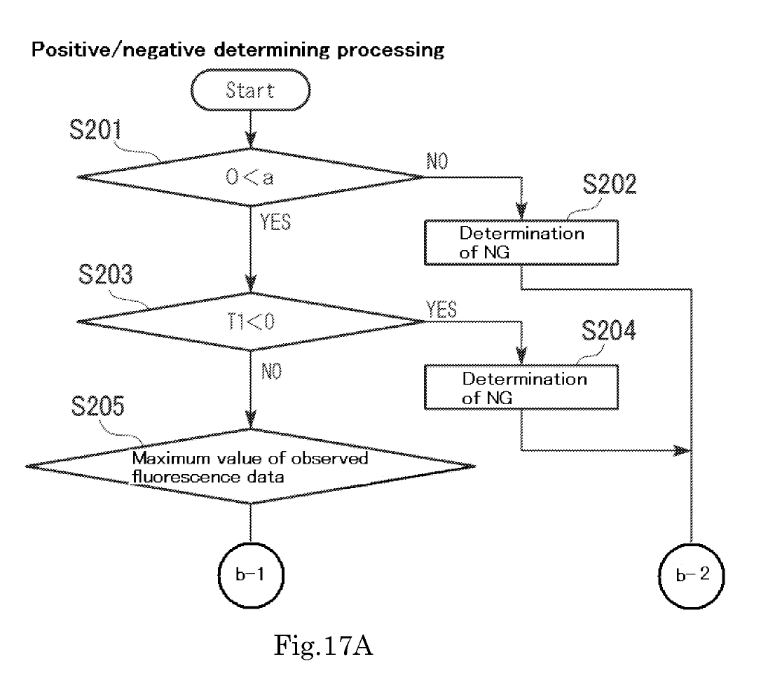

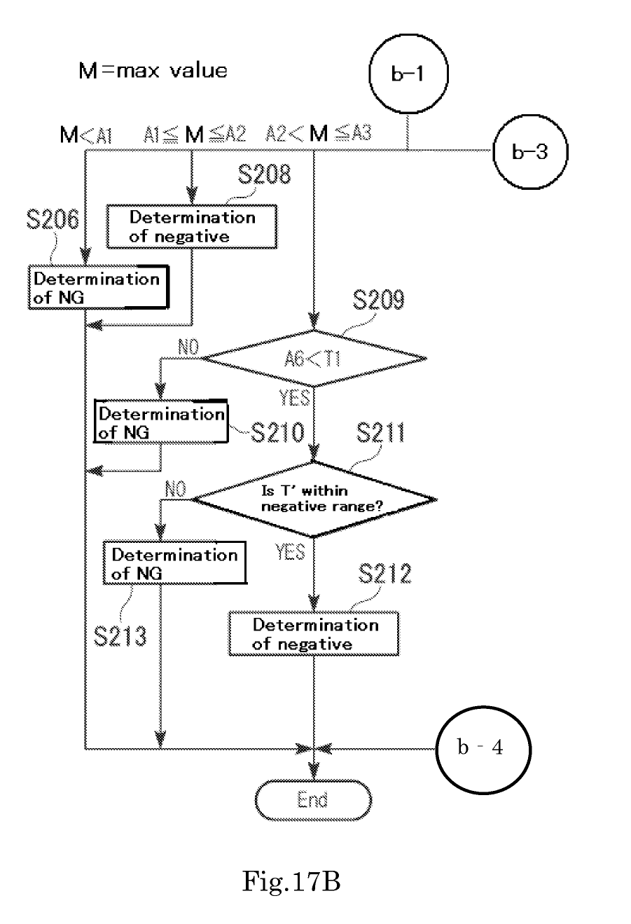

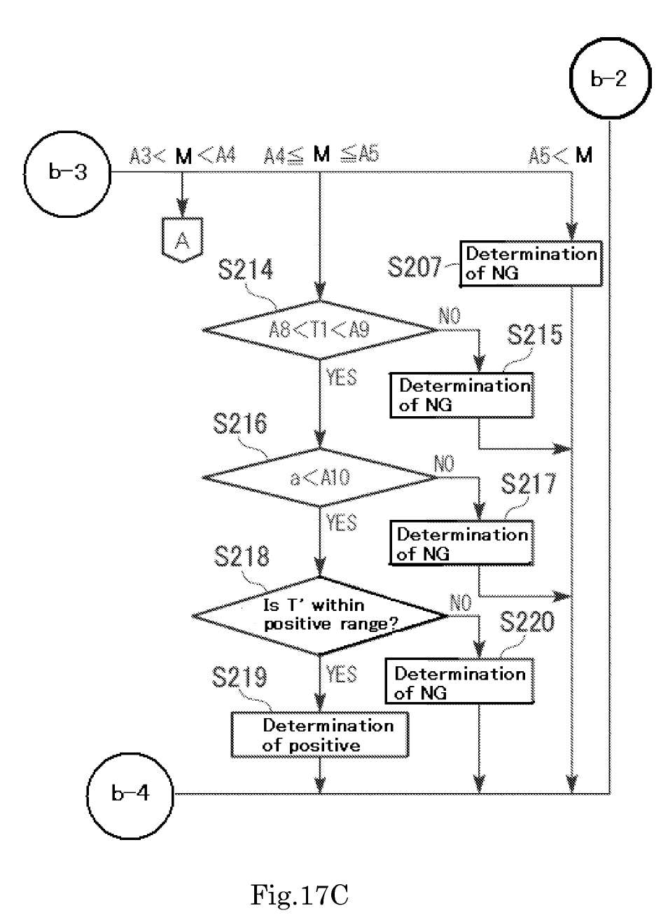

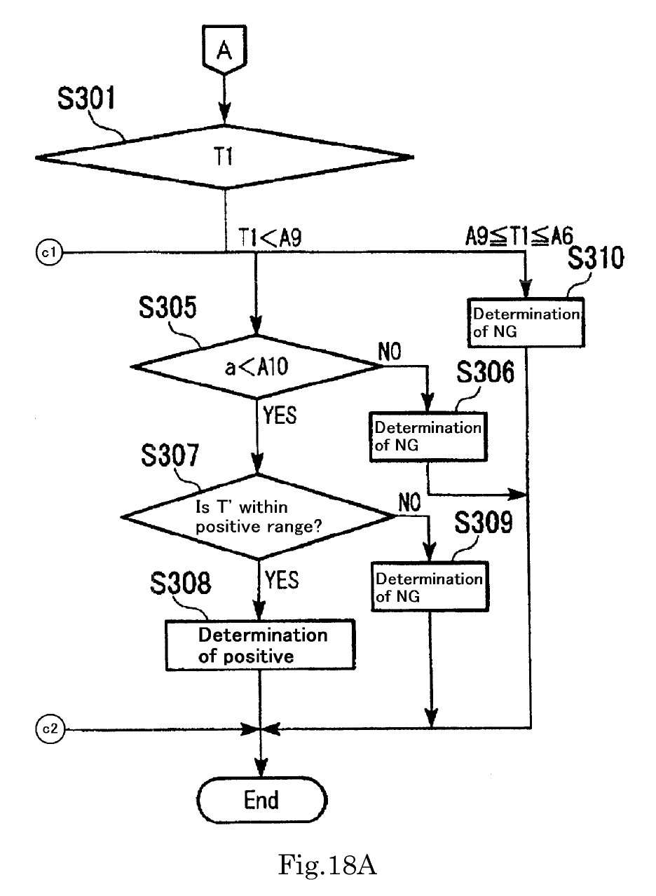

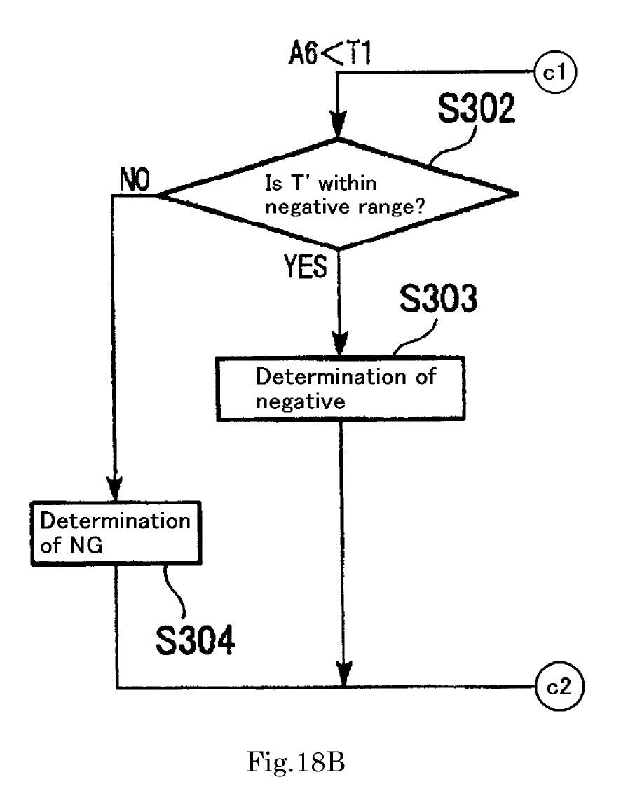

FIGS. 17 and 18 show positive/negative determining processing flow for obtaining the FAM determination result in step S102 and RED determination result in S103 in FIG. 15. A case in which FAM determination result is obtained will be described below, but this is the same as a case in which RED determination result is obtained and in that case, “FAM” should be replaced by “RED”. When the parameters a, b and c and the inflection point T1 are calculated by the logistic algorithm described in 2.2.1, FAM is parameters a^{AF}, b^{AF}, c^{AF}, inflection point T^{F}, and RED is parameters a^{AR}, b^{AR} , c^{AR} inflection nflection point T^{R}.

The determining unit 5 of the allele determining device 1 determines whether the parameter a of the approximate expression of FAM obtained in step S101 in FIG. 15 is greater than 0 (step S201). The parameter a is a value in which an amplification curve becomes constant, and substantially corresponds to a plateau value in reaction. When the parameter a in the approximate expression of the logistic curve of FAM is 0 or smaller (NO in step S201), it is determined that correct measurement could not be carried out, and the determining unit 5 determines that the positive/negative determination results of FAM are NG (step S202).

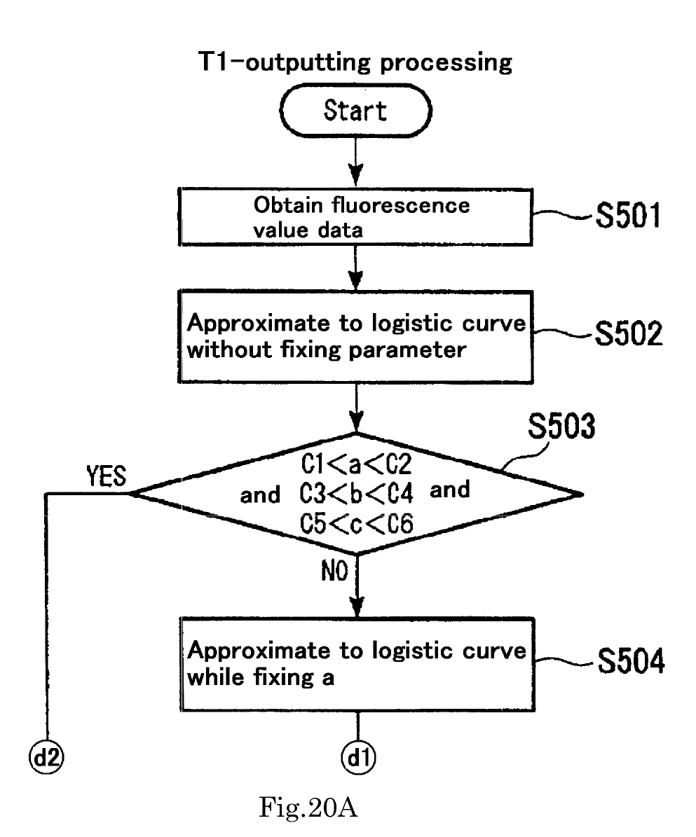

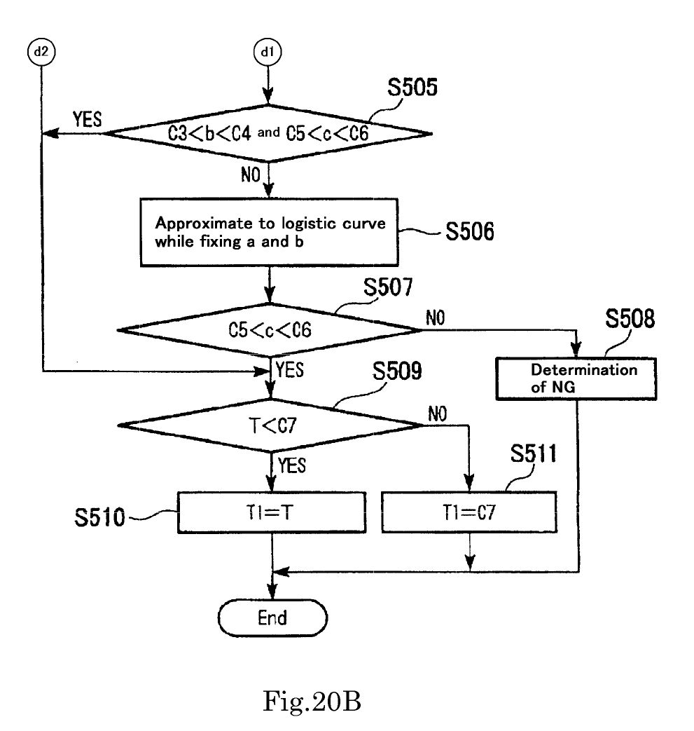

When the parameter a is greater than 0 (YES in step S201), the determining unit 5 obtains an inflection point T1 in an approximated logistic curve of FAM. The inflection point T1 is calculated by the logistic algorithm described in 2.2.1 or later-described T1 calculating processing in FIG. 20. The determining unit 5 determines whether the inflection point T1 is smaller than 0 (step S203). The inflection point T1 corresponds to reaction rising time. Hence, when the inflection point T1 is smaller than 0 (YES in step S203), the determining unit 5 determines that correct measurement could not be carried out and determines that the positive/negative determination results are NG (step S204).

When the inflection point T1 is 0 or greater (NO in step S203), the determining unit 5 branches by the maximum value (max value) of the measured value of the FAM fluorescence intensity (step S205).

The determining unit 5 determines that positive/negative determination results of FAM are NG (steps S206, S207) when the maximum value of the FAM fluorescence intensity that was actually observed for sample data of determination subject is abnormally small, i.e., when the maximum value is smaller than a threshold value A1 showing the minimum value of the FAM fluorescence intensity that is determined that normal measurement could be carried out (max value<A1 in step S205), or when the maximum value of the actually observed FAM fluorescence intensity is abnormally great, i.e., when the maximum value is greater than a threshold value A5 showing the maximum value of the FAM fluorescence intensity that is determined that normal measurement could be carried out (S5<max value in step S205).

When the maximum value of the FAM fluorescence intensity measured value is equal to or greater than the threshold value A1 and equal to or smaller than a threshold value A2 that is determined that FAM is negative (A1≦max value≦A2), the determining unit 5 determines that positive/negative determination results of FAM are negative (step S208). This is because that in an ideal case, a measured value of negative fluorescence intensity is sufficiently lower than a measured value of positive fluorescence intensity.

When the maximum value of the FAM fluorescence intensity measured value is greater than the threshold value A2 and equal to or smaller than a threshold value A3 that is determined that the possibility that FAM is negative is high, (A2<max value≦A3 in step S205), the determining unit 5 determines whether FAM is negative. In the case of positive, since reaction immediately rises, the inflection point T1 takes a small value, but in the case of negative, since reaction does not immediately rise, the inflection point T1 takes a value that is large to some extent. Hence, the determining unit 5 compares the inflection point T1 with a threshold value A6 for determining that FAM is negative (step S209). When the inflection point T1 is equal to or smaller than the threshold value A6(NO in step S209), the determining unit 5 determines that the FAM positive/negative determination results are NG (step S210).

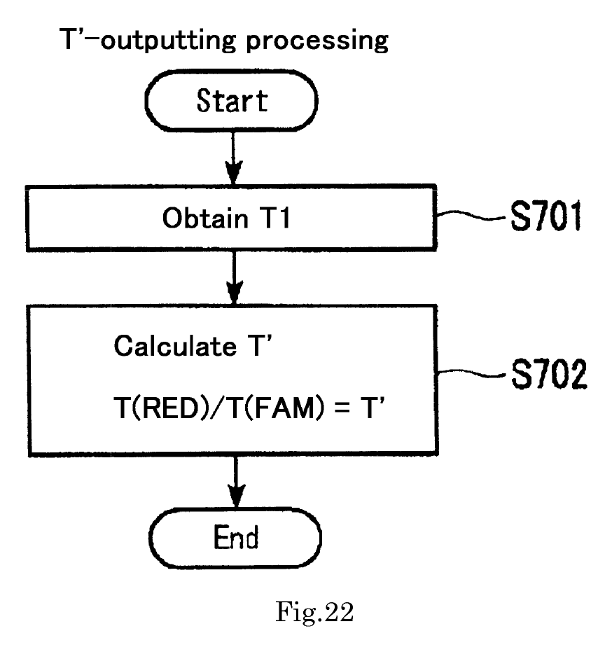

When the inflection point T1 is greater than the threshold value A6 (YES in step S209), the determining unit 5 calculates T′ that is a ratio of an inflection point T (FAM) of a logistic curve that is approximated from a measurement result of the FAM fluorescence intensity and an inflection point T (RED) of a logistic curve that is approximated from a measurement result of the RED fluorescence intensity by a later-described processing shown in FIG. 22. The determining unit 5 determines whether T′ is within a range where it is determined as negative (step S211). Here, it is determined whether T′ is smaller than a threshold value A7 that is determined that FAM is negative. When the determining unit 5 determines that T′ is within the range, i.e., when T′ is smaller than the threshold value A7 (YES in step S211), the determining unit 5 determines that the FAM positive/negative determination results are negative (step S212), and when T′ is not within the range, i.e., when T′ is equal to or greater than the threshold value A7 (NO in step S211), the determining unit 5 determines that the FAM positive/negative determination results are NG (step S213). In the case of RED, in order to determine whether T′ is within the range that is determined as negative, it is determined whether RED is greater than the threshold value A7 that is determined as negative.

The determining unit 5 further determines whether FAM is positive when the maximum value of the FAM fluorescence intensity measured value is equal to or greater than a threshold value A4 that is determined that possibility that the FAM is positive is high and when the maximum value is equal to or smaller than a above-described threshold value AS (A4≦max value≦A5 in step S205). In the case of positive, since reaction immediately rises, the inflection point T1 takes a small value. Hence, the determining unit 5 determines whether the inflection point T1 is greater than a threshold value A8 that is the minimum value for determining that FAM is positive and smaller than a threshold value A9 that is the maximum value for determining that FAM is positive (step S214). When the inflection point T1 is not a value existing between the threshold value A8 and the threshold value A9 (NO in step S214), the determining unit 5 determines that positive/negative determination results of FAM are NG (step S215).

When the inflection point T1 is the value existing between the threshold value A8 and the threshold value A9 (YES in step S214), the determining unit 5 determines whether the parameter a in the approximation curve of FAM is not an abnormally large value and whether the parameter a is smaller than a threshold value A10 that is determined that normal measurement was carried out (step S216). When it is determined that the parameter a is equal to or greater than the threshold value A10 (NO in step S216), the determining unit 5 determines that FAM positive/negative determination results are NG (step S217). When it is determined that the parameter a is smaller than the threshold value A10 (YES in step S216), the determining unit 5 determines whether the ratio T′ between the inflection point T (FAM) and the inflection point T (RED) calculated in later-described FIG. 22 are within a range that is determined as positive (step S218). Here, it is determined whether T′ is greater than a threshold value All that is determined that FAM is positive. When the determining unit 5 determines that T′ is within the range, that is, when the T′ is greater than the threshold value A11 (YES in step S218), it is determined that the FAM positive/negative determination results are positive (step S219). When it is determined that T′ is not within the range, i.e., when it is equal to or smaller than the threshold value A11 (NO in step S218), it is determined that the FAM positive/negative determination result are NG (step S220). In the case of RED, in order to determine whether T′ is within the range that is determined as positive, it should be determined whether RED is greater than the threshold value A11 that is determined as positive.

When the maximum value of the FAM fluorescence intensity measured value is greater than the threshold value A3 and smaller than the threshold value A4 (A3<max value<A4 in step S205), the determining unit 5 determines that there is possibility of both negative and positive, and determination shown in FIG. 18 is made.

In FIG. 18, the determining unit 5 branches by the inflection point T1 in the approximate expression of a logistic curve of the FAM (step S301). If the inflection point T1 is greater than a certain value, the possibility of negative is high. Hence, when the inflection point T1 is greater than a threshold value A6 (A6<T1 in step S301), the determining unit 5 determines whether the ratio T′ between the inflection point T (FAM) and the inflection point T (RED) is within a range that is determined as negative (step S302) under the same condition as that in step S211 shown in FIG. 17. When the determining unit 5 determines that the T′ is within the range (YES in step S302), it is determined that the FAM positive/negative determination results are negative (step S303), and when the determining unit 5 determines that T′ is not within the range (NO in step S302), it is determined that FAM positive/negative determination results are NG (step S304).

When the inflection point T1 is smaller than a certain value, the possibility of positive is high. When the inflection point T1 is smaller than a threshold value A9 for determining that FAM is positive (A9<T1 in step S301), the determining unit 5 carries out the same jobs as those in steps S216 to S220 in FIG. 17 (steps S305 to S309). That is, when it is determined that the parameter a of the approximation curve of FAM is equal to or greater than the threshold value A10 (NO in step S305), it is determined that FAM positive/negative determination results are NG (step S306). When it is determined that the parameter a is smaller than the threshold value A10 (YES in step S305), it is determined whether T′ is within a range that is determined as positive (step S307). When it is determined that T′ is within the range (YES in step S307), it is determined that the FAM positive/negative determination results are positive (step S308), and when it is determined that T′ is not within the range (NO in step S307), it is determined that the FAM positive/negative determination results are NG (step S309).

When the inflection point T1 is equal to or greater than the threshold value A9 and it is equal to or smaller than the threshold value A6 (A9≦T1≦A6 in step S301), the determining unit 5 determines that it is in a grey zone where it is not possible to determine whether it is positive or negative, and it is determined that the FAM positive/negative determination results are NG (step S310).

FIG. 19 is outputting processing of the parameter a of the allele determining device executed in step S101 in FIG. 15, that is executed for FAM and RED.

First, the approximating unit 4 reads, from the storing unit 3, a measurement result data of FAM fluorescence intensity F^{A}(t) of a sample of SNP determination subject in the case of FAM, and reads a measurement result data of RED fluorescence intensity R^{A}(t) of a sample of SNP determination subject in the case of RED (step S401). The approximating unit 4 uses the read data, approximates the data to logistic curves for FAM and RED by means of the method of least squares, and determines parameters in an approximate expression y=a/(1+be^{-cx}) (step S402). That is, the parameter a is a parameter a^{AF} in the case of FAM, and is a parameter a^{AR} in the case of RED.

The approximating unit 4 carries out the following processing for the parameter a of FAM and RED. That is, the approximating unit 4 compares the parameter a with a threshold value B1 that is the minimum value for determining whether the parameter a was correctly measured (step S403). When the parameter a is greater than the threshold value B1 (YES in step S403), the approximating unit 4 determines that the correct measurement was carried out, and outputs the parameter a to the determining unit 5 (step S404), and when the parameter a is it is equal to or smaller than the threshold value B1 (NO in step S403), the approximating unit 4 determines that the correct measurement was not carried out, and outputs FAM positive/negative determination NG to the determining unit 5 when a-output processing for FAM is being executed, and outputs RED positive/negative determination NG to the determining unit 5 when a-output processing for RED is being executed (step S405).

FIG. 20 shows output processing of T1 of the allele determining device that is executed in step S101 shown in FIG. 15, and this is executed for FAM and RED. Processing for FAM will be described below, but the processing for RED is also the same, and the processing may be executed while replacing “FAM” by “RED”.

The approximating unit 4 reads measurement result data of FAM fluorescence intensity F^{A}(t) of a sample of an SNP determination subject from the storing unit 3(RED fluorescence intensity R^{A}(T) in the case of RED) (step S501). The approximating unit 4 uses the read data, approximates the data to a logistic curve using the method of least squares, and determines parameters a, b and c in approximate expression y=a/(1+be^{-cx}) (step S502).

The approximating unit 4 determines whether the parameters a, b and c are within a range for determining whether correct measurement could be carried out (step S503). More specifically, it is determined whether the parameter a is between a threshold value C1 and a threshold value C2 that show a normal range in an approximation curve of FAM, and whether the parameter b is between a threshold value C3 and a threshold value C4 that show a normal range in the approximation curve of FAM, and whether the parameter c is between a threshold value C5 and a threshold value C6 that show a normal range in the approximation curve of FAM.

When any one or more of the parameters a, b and c are not within the range for determining whether the correct measurement could be carried out (NO in step S503), the approximating unit 4 fixes a to its value, uses the measured data shown by the FAM fluorescence intensity F^{A}(t) of a sample of the SNP determination subject, approximates the same to a logistic curve by the method of least squares or the like, and determines the parameters b and c (step S504). Here, the parameter a that is to be fixed is an average value of parameters a obtained by approximating time-varying fluorescence measured data to a logistic curve for each sample in which teacher data is FAM positive in a group in which a sample of measurement subject is not included. This group is a population for determining values of the parameters a and b that are to be fixed and other threshold values, and a human sample of the SNP determination subject should not belong to this population. The teacher data is data whose SNP genetic pattern (FAM Homo/Hetero/RED Homo) is already known by a sequencer or the like.

The approximating unit 4 determines whether the parameters b and c are within a range for determining whether the correct measurement could be carried out (step S505). More specifically, it is determined whether the parameter b is between the threshold value C3 and the threshold value C4 showing a normal range of an approximation curve of FAM, and whether the parameter c is between the threshold value C5 and the threshold value C6 showing the normal range of the approximation curve of FAM.

When one or both of the parameters b and c are not within the range for determining whether the correct measurement could be carried out (NO in step S505), the approximating unit 4 fixes the parameters a and b to their values, uses the measured data shown with the FAM fluorescence intensity F^{A}(t) of a sample of the SNP determination subject, approximates the data to a logistic curve by the method of least squares or the like, and calculates the parameter c (step S506). At that time, the parameter a to be fixed is a parameter a used in step S504, and the parameter b to be fixed is an average value of parameters b that are output by a later-described processing flow shown in FIG. 21.

The approximating unit 4 determines whether the parameter c is within a range for determining whether correct measurement could be carried out (step S507). More specifically, it is determined whether the parameter c is between the threshold value C5 and threshold value C6. When the parameter c is not within the range for determining whether correct measurement could be carried out (NO in step S507), the approximating unit 4 outputs FAM positive/negative determination NG to the determining unit 5 (step S508).

When it is determined in step S503 that all of the parameters a, b and c are within the range for determining whether correct measurement could be carried out (YES in step S503), when it is determined in step S505 that both the parameters b and c are within the range for determining whether correct measurement could be carried out (YES in step S505), and when it is determined in step S507 that the parameter c is within the range for determining whether correct measurement could be carried out (YES in step S507), the approximating unit 4 calculates the inflection point T using the calculated parameters a, b and c. The approximating unit 4 determines whether the inflection point T of FAM is within a range for determining it appears within predetermined time (step S509). For example, the approximating unit 4 determines whether the inflection point T is smaller than the threshold value C7. When the inflection point T is smaller than the threshold value C7 (YES in step S509), the inflection point T1 used in the positive/negative determination flow in FIGS. 17 and 18 is output to the determining unit 5 as an inflection point T obtained from the approximate expression of a logistic curve of FAM (step S510). That is, an inflection point T1=T (FAM) is output in the case of FAM, and an inflection point T1=T (RED) is output in the case of RED. When the inflection point T is equal to or greater than the threshold value C7 (NO in step S509), the inflection point T1 used in the positive/negative determination flow in FIGS. 17 and 18 is used as the threshold value C7 (step S511).

FIG. 21 shows an outputting processing flow for determining a parameter b to be fixed. This processing is executed for each of samples in which teacher data is FAM positive for the above-described group in which a sample of a measurement subject is not included. An average value of parameters b calculated in this processing is a parameter b to be fixed in step S506 in FIG. 20.

The approximating unit 4 reads measurement result data of FAM fluorescence intensity F^{A}(t) (RED fluorescence intensity R^{A}(t) in the case of RED) of a sample in which teacher data is FAM positive included in the group in which a sample of a measurement subject is not included (step S601). The approximating unit 4 uses the measurement result data obtained in step S601, approximates the same to a logistic curve without fixing a parameter by the method of least squares or the like for the sample, and calculates the parameters a, b and c in the approximate expression y=a/(1+be^{-cx}) (step S602).

The approximating unit 4 determines whether the parameters a, b and c calculated in step S602 are within a range for determining whether correct measurement could be carried out (step S603). More specifically, the approximating unit 4 determines whether the parameter a is between the threshold value Cl and the threshold value C2 showing a normal range in the approximation curve of FAM, and whether the parameter b is between the threshold value C3 and the threshold value C4 showing a normal range in the approximation curve of FAM, and whether the parameter c is between the threshold value C5 and the threshold value C6 showing a normal range in the approximation curve of FAM. When all of the calculated parameters a, b and c are within the range for determining whether correct measurement could be carried out (YES in step S603), the approximating unit 4 outputs the calculated parameter b (step S605).

When one or more of the parameters a, b and c are not within the range for determining whether correct measurement could be carried out (NO in step S603), the approximating unit 4 fixes a, uses measured data of a sample that is read in step S601, approximates the same to a logistic curve by the method of least squares or the like, determines the parameter b (step S604), and outputs the determined parameter b (step S605). Here, the parameter a that is to be fixed is an average value of parameters a obtained by approximating time-varying fluorescence measured data to a logistic curve for each sample in which teacher data is FAM positive in a group in which a sample of measurement subject is not included.

FIG. 22 is a calculation flow of T′ used in the positive/negative determination flow in FIGS. 17 and 18. The approximating unit 4 uses the inflection points T^{F} and T^{R} calculated in 2.2.1, and T1 of FAM and RED obtained in FIG. 20 as T (FAM) and T (RED) (step S701), calculates T′=T (RED)/T (FAM), and outputs the same to the determining unit 5 (step S702).

The threshold values A1 to A11, B1 and C1 to C7 depend on measuring conditions such as temperature and an amount of specimens. Thus, an appropriate value is determined from a statistic of actually measured data, and that value is stored in the storing unit 3.

[4. Result of Experiment]

A result of an experiment using the SNP determination by the conventional end point method, and the SNP determination in which determination was carried out by the flow by the allele determining device 1 of the embodiment is shown below.

[4.1 Method of Experiment] [4.1.1 End Point Method Algorithm]

In the end point method described in the conventional technique, padding is subtracted from corrected data, and negative control is used for matching scales of FAM and RED with each other, but the negative control is not used in this demonstration experiment. Thus, padding is not subtracted from the corrected data described below. To match the scales with each other, RED having low fluorescence value is multiplied by an arbitrary value instead of using the negative control, thereby adjusting the fluorescence values of FAM and RED.

Data and algorithm used in the experiment including changed points from the method described in the conventional technique will be described below.

(1) Data: sample data (raw data): FAM sample data F^{A}(t) and RED sample data R^{A}(t) that are fluorescence intensities of FAM and RED after t-minutes (measured values of device) are obtained.

(2) Corrected data: F^{R}(T) and R^{R}(T) after T-minutes are obtained.

FAM: F^{R}(T)=F^{A}(T)

RED: R^{R}(T)=h×R^{A}(T)

wherein, h represents a parameter for matching scales.

(3) Algorithm

A ratio of the corrected data Ratio is calculated, and allele is determined.

Ratio=F^{R}(T)/R^{A}(T)

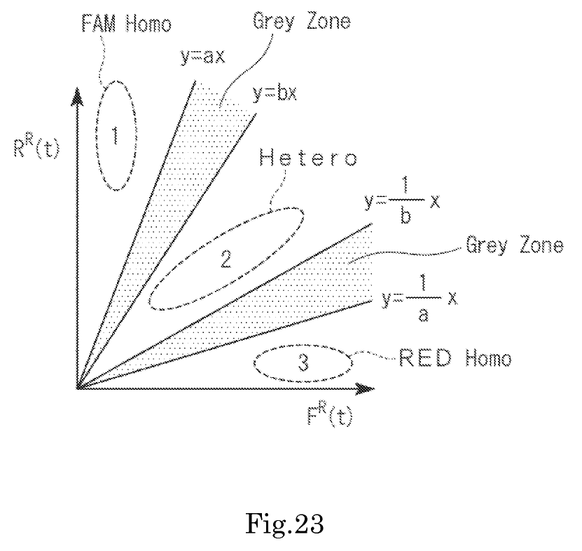

As shown in FIG. 23, grey zones were set to b<(grey zone)<a and (1/a)<(grey zone)<(1/b), RED Homo was set to Ratio<(1/a), Hetero was set to (1/b)<Ratio <b, and FAM Homo was set to a<Ratio, and determination was made.

[4.1.2 Logistic Algorithm]

An experiment was carried out by substantially the same method as that of the processing flow described in 3. Details of kinds of data used in the experiment, and formation of algorithm will be described below.

(1) The following time-series data is obtained.

Sample data (raw data): FAM sample data F^{A}(t) and RED sample data R^{A}(t) that were fluorescence intensities (measured value of the allele determining device 1) of FAM and RED after t-minutes were obtained.

(2) The sample data is approximated to a logistic curve, and the following parameters are obtained.

Sample FAM: a^{AF}, b^{AF} and C^{AF} that are parameters a, b and c calculated using the FAM sample data F^{A}(t)

Sample RED: a^{AR}, b^{AR}, c^{AR} that are parameters a, b and c calculated using the RED sample data R^{A}(t)

(3) An inflection point of a logistic curve that was applied in (2) is calculated.

Inflection point of FAM: T^{F}=(logb^{AF})/c^{AF}

Inflection point of RED: T^{R}=(logb^{AR})/c^{AR}

(4) Determination is made while utilizing the inflection points T^{F} and T^{R} calculated in (3).

(5) Calculates the ratio T′ of the inflection points T^{F} and T^{R}.

Inflection point ratio: T′=T^{R}/T^{F}

(6) The maximum value of sample data is obtained.

Maximum value of FAM sample data: M^{F}

Maximum value of RED sample data: M^{R}

(7) Determination is made in accordance with the determination flow while utilizing the maximum values M^{F} and M^{R}, parameters a^{AF} and a^{AR} of logistic curve, and the ratio T′ of the inflection points T^{F} and T^{R}, in addition to the inflection points T^{F} and T^{R}.

[4.2 Result]

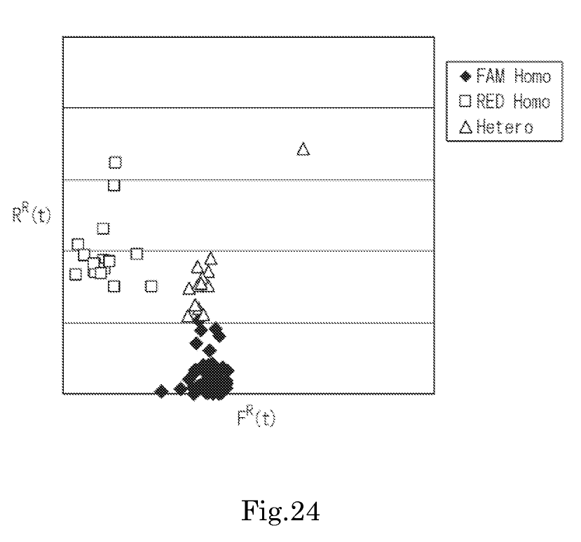

FIG. 24 shows a graph of a fluorescence value in the end point algorithm wherein a horizontal axis shows F^{R}(T) and a vertical axis shows R^{R}(T). The genetic patterns (FAM Homo/Hetero/RED Homo) of SNP shown here are teacher data. In the drawing, in the end point algorithm, there are portions where plots of Homo and Hetero are superposed on each other. Plots of NG and normal data are superposed on each other in some cases, and it can be found that it is extremely difficult to set threshold when algorithm is formed.

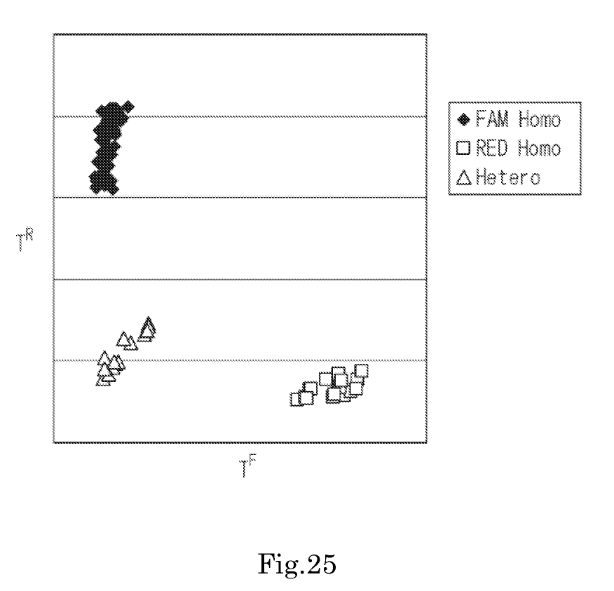

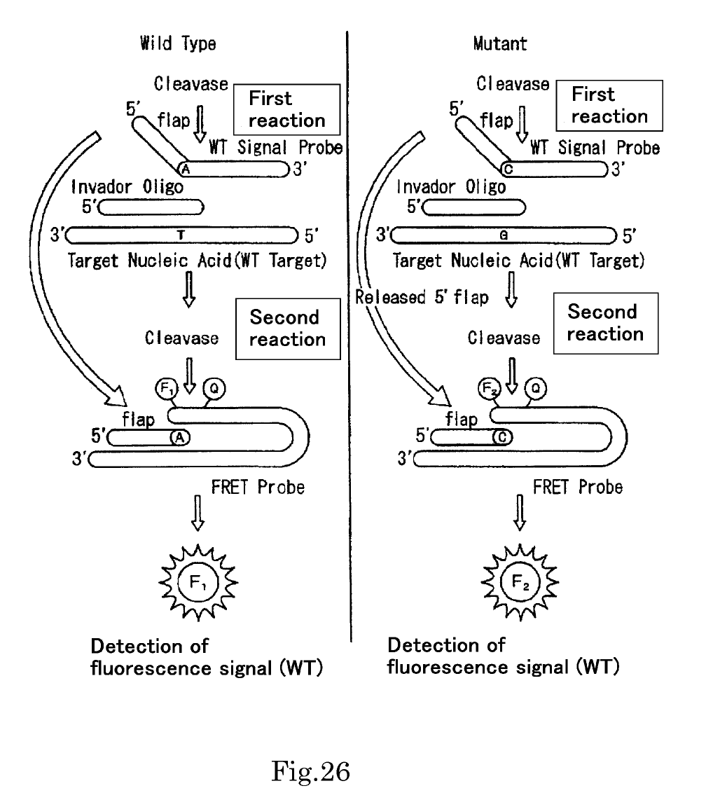

FIG. 25 shows correlation of the inflection points T^{F} and T^{R} when logistic algorithm is used. The drawing is a graph where a horizontal axis shows the inflection point T^{F} of RED and the vertical axis shows the inflection point T^{R} of FAM. The genetic patterns (FAM Homo/Hetero/RED Homo) of the SNP are determined by the teacher data. As shown in the drawing, there is no portion where plots of Home and Hetero are superposed on each other, and the plots are in a cluster.

Determination results by the teacher data and logistic algorithm matched with each other with high precision, and erroneous determination was not found in this experiment.

[5. Others]

Although approximation to a logistic curve is carried out in the above description, other curves such as Gompertz curve may be used. The Gompertz curve is an S-shaped curve, and is given by the following equation.

f(x)=ab^{C}^{^{x}} [Equation 3]

Here, a is given by the following equation.

$$a = {lim}_{t -> ∞} f ( x ) \qquad [ Equation 4 ]$$

Here, b and c fall within the following ranges.

0<b<1 and 0<c<1

Points that correspond to the inflection point T of a logistic curve are as follows.

$$T = ( \frac{- ln - ln b}{ln c} , \frac{a}{e} ) \qquad [ Equation 5 ]$$

The allele determining device 1 includes a computer system therein. The processes of operation of the approximating unit 4, the determining unit 5 and the outputting unit 6 of the allele determining device 1 are stored in a storing medium that can be read by a computer in a form of a program, and the above processing is carried out by reading and executing the program by the computer system. The computer system includes a CPU, various memories, OS, and hardware such as peripheral devices.

If the “computer system” utilizes a WWW system, it includes a home page supply environment (or display environment).

Further, the “storing medium that can be read by a computer” includes a transportable medium such as a flexible disk, a magneto-optic disk, a ROM and a CD-ROM, and a storing device such as a hard disk drive incorporated in the computer system. Furthermore, the “storing medium that can be read by a computer” includes a medium that dynamically holds a program for a short time such as a communication line when the program is sent through a network such as the Internet and a communication line such as a telephone line, and includes a medium that holds the program for a given period of time such as a volatile memory in the computer system that becomes a server or client in that case. The program may realize a portion of the above-described function, or may realize the function in combination with a program that is already stored in the computer system.

According to the inventions of the first aspect, the fourteenth aspect and the sixteen aspect, since it is possible to determine the single nucleotide polymorphism based on variation in intensity of light with time instead of based on a result of temporary optical measurement of a reagent that reacts with a specific base sequence of gene unlike the conventional technique, it is possible to enhance the precision of determination.

According to the second aspect of the invention, since it is possible to determine the single nucleotide polymorphism based on time when intensity of observed light is varied, it is possible to further enhance the precision of determination.

According to the third aspect of the invention, since it is possible to determine the single nucleotide polymorphism by an index of the maximum value of intensity of light indicated by an approximated curve, it is possible to further enhance the precision of determination.

According to the fourth aspect of the invention, since it is possible to determine the single nucleotide polymorphism by the maximum value of intensity of observed light, it is possible to further enhance the precision of determination.

According to the fifth aspect of the invention, the optical measurement results using two kinds of reagents that react with different specific base sequences can be approximated to the predetermined curve, and it can be determined whether the reaction of each of the reagents is positive or negative using the characteristic point of the curve, and the single nucleotide polymorphism can be determined by the determination result.

According to the sixth aspect of the invention, when the end point method is used for determining the single nucleotide polymorphism, it is possible to find the optimal end point time.

According to the seventh aspect of the invention, subject performance of plots is enhanced, and clustering is facilitated as compared with the conventional end point method in which plots of a ratio of the fluorescence value of the two kinds of regents (FAM, RED) are clustered by straight line equations y=ax and y=(1/a)x and a determination result is obtained.

According to the eighth aspect, the fifteen aspect and the seventeen aspect of the inventions, it is possible to determine the single nucleotide polymorphism based on variation in the time-varying light intensity instead of optical measurement result of a reagent that reacts with a specific base sequence of a gene at one time point of the conventional technique. Therefore, it is possible to enhance the precision of determination.

According to the ninth aspect of the invention, it is possible to determine the single nucleotide polymorphism based on a difference of varying speed of light intensity of the two kinds of reagents.

According to the tenth aspect of the invention, it is possible to determine the single nucleotide polymorphism based on a point at which a difference of varying speed of light intensity of the two kinds of reagents becomes the maximum.

According to the eleventh aspect of the invention, since the curve to be approximated is a logistic curve, it is possible to obtain a characteristic point that can easily be utilized for determining the single nucleotide polymorphism.

According to the twelfth aspect of the invention, the device can be used for determining the single nucleotide polymorphism by the fluorescence reaction using a probe that reacts with a specific base sequence.

According to the thirteenth aspect of the invention, the device can be used for determining the single nucleotide polymorphism by the Invader (registered trademark) method.

1. An allele determining device that determines a single nucleotide polymorphism of a gene, comprising:
approximating means that approximates an optical measurement result obtained by observing a reagent that reacts with a specific base sequence of a gene, to a predetermined curve using light intensity and time as parameters; and
determining means that determines a single nucleotide polymorphism using a characteristic point of the predetermined curve that was approximated by the approximating means.

2. The allele determining device according to claim 1, wherein the characteristic point is an inflection point in the predetermined curve that was approximated by the approximating means.

3. The allele determining device according to claim 2, wherein the determining means determines the single nucleotide polymorphism using an index of a maximum value of the light intensity in the predetermined curve that was approximated by the approximating means.

4. The allele determining device according to claim 2, wherein the determining means determines the single nucleotide polymorphism using a maximum intensity of observed light indicated by the optical measurement result.

5. The allele determining device according to claim 1, wherein the approximating means approximates optical measurement results of two kinds of reagents that react with different specific base sequences to the predetermined curve, and
the determining means determines whether reactions of the reagents are positive or negative using the characteristic point of the predetermined curve that was approximated by the approximating means for each of the reagents, and determines the single nucleotide polymorphism from the determination of whether the reactions of the reagents are positive or negative.

6. The allele determining device according to claim 1, wherein the determining means calculates an endpoint time from the characteristic point, and determines the single nucleotide polymorphism using the optical measurement result observed at the calculated end point time.

7. The allele determining device according to claim 6, wherein the determining means determines the single nucleotide polymorphism further using a logarithm of a ratio of the optical measurement result at the calculated end point time of each of two kinds of reagents that react with different specific base sequences.

8. The allele determining device according to claim 1, wherein the characteristic point of the predetermined curve that was approximated by the approximating means is a characteristic point obtained from a logarithm of the predetermined curve that was approximated by the approximating means.

9. The allele determining device according to claim 8, wherein the approximating means approximates optical measurement results of two kinds of reagents that react with different specific base sequences to the predetermined curve, and
the determining means determines the single nucleotide polymorphism using a characteristic point obtained from a logarithm of a ratio of the predetermined curve that was approximated to the reagents by the approximating means.

10. The allele determining device according to claim 9, wherein the characteristic point is a peak value in the logarithm of the ratio.

11. The allele determining device according to claim 1, wherein the predetermined curve is a logistic curve.

12. The allele determining device according to claim 1, wherein the optical measurement result is a measured value of a fluorescence reaction using a probe that reacts with the specific base sequence.

13. The allele determining device according to claim 12, wherein the fluorescence reaction is includes a reaction process using a substrate specificity of an enzyme.

14. An allele determining method for determining a single nucleotide polymorphism of a gene, comprising:
an approximating step of approximating an optical measurement result obtained by observing a reagent that reacts with a specific base sequence of a gene, to a predetermined curve using light intensity and time as parameters; and
a determining step of determining a single nucleotide polymorphism using a characteristic point of the predetermined curve that was approximated in the approximating step.

15. The allele determining method according to claim 14, wherein the characteristic point of the predetermined curve that was approximated in the approximating step is a characteristic point obtained from a logarithm of the predetermined curve that was approximated in the approximating step.

16. A computer program, wherein a computer used as an allele determining device that determines a single nucleotide polymorphism of a gene functions as
approximating means that approximates an optical measurement result obtained by observing a reagent that reacts with a specific base sequence of a gene, to a predetermined curve using light intensity and time as parameters, and
determining means that determines a single nucleotide polymorphism using a characteristic point of the predetermined curve that was approximated by the approximating means.

17. The computer program according to claim 16, wherein the characteristic point of the predetermined curve that was approximated by the approximating means is a characteristic point obtained from a logarithm of the predetermined curve that was approximated by the approximating means.

18. An allele determining device according to claim 1,
wherein the number of the single nucleotide polymorphism is two or more, and the number of the gene is two or more,
comprising
approximating means that approximates an optical measurement result obtained by observing a reagent that reacts with a specific base sequence of each of the genes, to a predetermined curve using light intensity and time as parameters, and
determining means that determines each of the single nucleotide polymorphisms using a characteristic point of the curve that was approximated by the approximating means.
